(19) United States
(12) Patent Application Publication
(10) Pub. No.: US 2024/0350567 A1
(43) Pub. Date: Oct. 24, 2024

(54) USE OF LACTOBACILLUS PARACASEI BACTERIAL STRAINS IN THE TREATMENT OF NEWBORNS

(71) Applicant: Alfasigma S.p.A., Bologna (IT)

(72) Inventors: Andrea Biffi, Bologna (IT); Walter Fiore, Bologna (IT)

(73) Assignee: Alfasigma S.p.A., Bologna (IT)

(21) Appl. No.: 18/579,158

(22) PCT Filed: Jul. 15, 2022
(86) PCT No.: PCT/IB2022/056538

§ 371 Date: Jan. 12, 2024

(30) Foreign Application Priority Data
Jul. 15, 2021 (IT) 102021000018740

(51) Int. Cl. A61K 35/747; A61K 35/745; A61K 45/06; A61P 1/00

(52) U.S. Cl. CPC A61K 35/747; A61K 35/745; A61K 45/06; A61P 1/00

(57) ABSTRACT

The present invention relates to at least one strain of bacteria belonging to the species Lactobacillus paracasei, preferably Lactobacillus paracasei DG® CNCM I-1572 and/or Lactobacillus paracasei LPC-01 DSM 26760, and compositions thereof for use in newborns and/or from 1 month to ≤12 months subjects in the treatment, preventive and/or curative, of gastro-intestinal tract disorders of an inflammatory and/or functional nature, gastro-intestinal infections by pathogenic micro-organisms, gastro-intestinal infections by parasites, allergies, immune-mediated or autoimmune disorders, and in the support of the subject's growth.

The present invention relates to at least one strain of bacteria belonging to the species Lactobacillus paracasei, preferably Lactobacillus paracasei DG® CNCM I-1572 and/or Lactobacillus paracasei LPC-S01 DSM 26760, and compositions thereof for use in newborn subjects in the first 4 weeks of life in the treatment, preventive and/or curative, of gastro-intestinal tract disorders of an inflammatory and/or functional nature, preferably neonatal colic or chronic enterocolitis in preterm newborns (e.g. necrotizing enterocolitis), in the treatment, preventive and/or curative, of gastro-intestinal infections by pathogenic microorganisms (e.g. viruses or bacteria), gastro-intestinal infections by parasites, allergies, immune-mediated or autoimmune disorders, and in the supportive treatment of the subject's growth.

Microbial colonisation of the newborn intestine begins immediately after birth and is essential for the development of the mucosal barrier function, intestinal homeostasis and the maturation of the immune system.

In the context of the present invention, the term ‘newborn’ or ‘newborn subject’ refers to a mammal (human or animal subject) in the period between the moment of birth and the first 4 weeks (28 days) of life.

In the first days of a baby's life, numerous factors influence the composition of the gut microbiota: the mother's vaginal and/or skin microbiota, vaginal or caesarean delivery, breast milk (breastfeeding) or artificial milk feeding, and the administration of antibiotics and/or other drugs. In particular, caesarean section, artificial milk, preterm delivery and the use of antibiotics reduce the abundance and diversity of beneficial bacterial species in the microbiota, promoting a state of dysbiosis. This in turn may increase the risk of the onset of intestinal colic, necrotic enterocolitis in preterm newborns and the development of immune-mediated diseases later in life, such as allergies or inflammatory or functional bowel disease, and/or related symptoms.

Currently, about 12% of preterm newborns weighing less than 1,500 g suffer from necrotic enterocolitis and about one third die from sepsis or other complications.

Newborns are not physiologically comparable to ‘fragile’ adults, nor even to very young children, because in the first weeks of life the maturation of internal organs is not complete and their consequent functionality is not comparable to that of a child or an adult, however debilitated or ill they may be.

‘Gut microbiota’ refers to all microorganisms (all bacteria, archaea, eukaryotes, and viruses) present in the gastro-enteric (or gastro-intestinal) environment.

The microbiota performs many useful activities for the host organism. Indeed, the microbes that compose it degrade polysaccharides, aiding digestive functions, synthesize vitamins, hinder colonisation by pathogenic species, provide signals necessary for intestinal development, and contribute to modulate inflammatory and immune responses. The aetiology of many gastro-enteric disorders, as well as those affecting other systems, is believed to be an aberrant immunological response to gut microbes. Moreover, the gut microbiota constitutes, together with the metabolites it produces and enteric cells, a barrier, the malfunctioning of which is implicated in the pathophysiology of many immune-mediated disorders as well as infections by pathogenic microorganisms.

It is necessary, in order to maintain a good state of health, that there is a balance between the immune system and the microbes residing in the gut. This state of balance has therefore also been shown to depend on the composition of the gut microbiota: imbalances in the microbiota, known under the term ‘dysbiosis’, can lead to immune system disorders, which cause harmful effects both in the gut and in other systems.

The beneficial effects of probiotic bacterial strains (i.e. live microorganisms that, when administered in adequate amounts, confer health benefits) are therefore mainly attributed to normalising the permeability of the intestinal barrier, regularising the intestinal microbiota and restoring the balance between the microbiota and the immunological response.

The technical problem which the present invention addresses and solves is to provide an effective solution for the treatment of gastro-intestinal diseases or symptoms of an inflammatory or functional nature (for example: intestinal colic, chronic enterocolitis) and gastro-intestinal infections by pathogenic micro-organisms or gastro-intestinal infections by parasites in newborn subjects and/or mammalian subjects in the first months of life (from 1 month to ≤12 months; a month may have 28, or 29, or 30, or 31 days).

Furthermore, the present invention addresses and solves the technical problem of preventively treating the onset of allergies and/or immune-mediated or autoimmune disorders related to the gastrointestinal system (e.g. coeliac disease) in the later years of life of the treated subject. Finally, it is an object of the present invention to provide an effective solution for supporting the body growth of newborns and/or mammalian subjects in the first months of life.

The Applicant, following research and development activity, addresses and solves the aforementioned technical problem by providing at least one strain of bacteria belonging to the species Lactobacillus paracasei, such as Lactobacillus paracasei DG® CNCM I-1572 and/or Lactobacillus paracasei LPC-S01 DSM 26760, and compositions thereof (in short, compositions of the invention) that effectively and efficiently contribute to the development of an appropriate and lactobacillus-rich, in particular Lactobacillus paracasei, gut microbiota in the newborn and/or subject in the first months of life.

Among the strains of bacteria of the genus Lactobacillus, the Lactobacillus paracasei species (such as Lactobacillus paracasei strain DG® CNCM I-1572 and/or Lactobacillus paracasei strain LPC-S01 DSM 26760) belongs to the species commonly used as probiotic bacteria strains. In particular, the L. paracasei strain DG® CNCM I-1572 has been extensively studied for its beneficial properties. However, the effects of L. paracasei DG® CNCM I-1572 on newborn subjects and/or subjects from 1 month to ≤12 months have never been studied to date.

It should to be noted that the strain of bacteria L. casei DG® (CNCM I-1572) or L. paracasei DG® (CNCM I-1572) was redeposited on 2 Feb. 2022 as Lacticaseibacillus paracasei DG I-1572 DSM 34154 following the reclassification of the genus Lactobacillus published by Zheng et al. on the scientific journal Int. J. Syst. Evol. Microbiol., 70 (4): 2782-2858, 2020. The two aforementioned names are interchangeable with each other because they always refer to the same strain of bacteria.

L. paracasei strain DG® CNCM I-1572 and L. paracasei strain LPC-S01 DSM 26760, mixtures and compositions thereof according to the present invention administered to subjects in the neonatal age (first 4 weeks of life) appear to be able to effectively and efficiently colonise the intestinal tract during the period of administration and/or the first months of life, leading to a balance between the intestinal microbiota and the immune system beneficial to the host, and to the strengthening of the permeability of the intestinal barrier.

Finally, the strains of bacteria, mixtures and compositions thereof of the present invention are well tolerated, have no relevant side effects, are easy to prepare and cost-effective.

These purposes and others, which will become clear from the detailed description below, are achieved by the compositions and mixtures of the present invention due to the technical features claimed in the appended claims.

### FIGURES

FIG. 1: scheme of the clinical study;

FIGS. 2A to 2E: quantification of L. casei DG® in arm 1 (composition under analysis; Tx) and arm 2 (Placebo) in faecal samples collected during V1-V5 control visits;

FIGS. 3A to 3D: Histograms of the relative abundance of Lactobacillus spp. at time points of V1-V5 control visits;

FIG. 4: Metabolomic predominance of partial least squares-discriminant analysis (PLS-DA) showing the identified metabolites, including butanoic acid and propanoic acid.

### DETAILED DESCRIPTION OF THE INVENTION

It is an object of the present invention at least one strain of bacteria belonging to the species Lactobacillus paracasei, preferably the Lactobacillus paracasei strain DG® CNCM I-1572 and/or the Lactobacillus paracasei strain LPC-S01 DSM 26760, mixtures or compositions thereof as defined hereinafter of the present invention (in short, mixtures or compositions of the invention) for use in newborn subjects (first 4 weeks of life) and/or subjects from 1 month to ≤12 months in the treatment, preventive and/or curative, of gastro-intestinal tract disorders of an inflammatory and/or functional nature such as neonatal colic or intestinal colic, chronic enterocolitis or chronic enterocolitis in preterm newborns, necrotising enterocolitis, sepsis, irritable bowel syndrome (IBS for short), IBS with prevalent constipation or constipated bowel, IBS with prevalent diarrhoea or diarrhoeic bowel, IBS with alternating bowel, unclassified IBS, or chronic inflammatory bowel disease (IBD), such as Crohn's disease, ulcerative rectocolitis, asthma, obesity, type 1 diabetes, atopic dermatitis, multiple sclerosis, cancer, and autism.

Preferably, the strains, mixtures or compositions thereof of the present invention are for use in nweborn subjects and/or subjects from 1 month to ≤12 months in the treatment, preventive and/or curative, of neonatal colic or chronic enterocolitis in preterm newborns or necrotizing enterocolitis.

It is an object of the present invention to at least one strain of bacteria belonging to the species Lactobacillus paracasei, preferably the Lactobacillus paracasei strain DG® CNCM I-1572 and/or the Lactobacillus paracasei strain LPC-S01 DSM 26760, mixtures or compositions thereof as defined below of the present invention (in short, mixtures or compositions of the invention) for use in newborn subjects (first 4 weeks of life) and/or subjects from 1 month to ≤12 months in the treatment, preventive and/or curative, of gastro-intestinal infections by pathogenic microorganisms (e.g. viruses (such as Rotavirus, Adenovirus entericus, Calicivirus, Astrovirus, influenza virus), or bacteria such as Salmonella, Shigella, Staphylococcus, Campylobacter, Escherichia coli), gastro-intestinal infections by parasites (preferably caused by helminths or intestinal worms (e.g. Ossiuri, Giardia, etc.).), allergies, immune-mediated or autoimmune disorders (e.g. coeliac disease, Graves' disease, rheumatoid arthritis, Hashimoto's thyroiditis, type 1 diabetes mellitus, systemic lupus erythematosus (lupus), vasculitis, Addison's disease, polymyositis, Sjögren's syndrome, progressive systemic sclerosis, glomerulonephritis (inflammation of the kidneys), infertility, asthma, obesity, type 1 diabetes, atopic dermatitis, multiple sclerosis, cancer, and autism.

It is an object of the present invention at least one strain of bacteria belonging to the species Lactobacillus paracasei, preferably the Lactobacillus paracasei strain DG® CNCM I-1572 and/or the Lactobacillus paracasei strain LPC-S01 DSM 26760, mixtures or compositions thereof as defined hereinafter of the present invention (in short, mixtures or compositions of the invention) for use in newborn subjects (first 4 weeks of life) and/or subjects from 1 month to ≤12 months in the supportive (adjuvant) treatment of subject's growth, understood as growth in body weight of the subject administered, assessed as g/week.

The strain of bacteria identified as Lactobacillus paracasei DG® (registered trademark by SOFAR S.p.A., Italy) was deposited by SOFAR S.p.A. at the National Collection of Cultures of Microorganisms of the Institut Pasteur in Paris under the accession number CNCM I-1572 on 5 May 1995 by SOFAR S.p.A. (in short, DG® or L. paracasei DG® CNCM I-1572); initially the strain had the name Lactobacillus casei DG® sub.casei; it was later reclassified as Lactobacillus paracasei DG® CNCM I-1572. It is clarified that it is always and only the same strain of bacteria, regardless of the designation Lactobacillus casei DG® or Lactobacillus paracasei DG®.

The strain of bacteria identified as Lactobacillus paracasei LPC-S01, alternatively named Lactobacillus paracasei S01, was deposited at the Deutsche Sammlung von Mikroorganismen und Zellkulturen GmbH (DSMZ) under the accession number DSM 26760 on 20 Nov. 2012 by SOFAR S.p.A. (in short, LPC-S01 or L paracasei LPC-S01 DSM 26760). It is clarified that it is always and only the same strain of bacteria regardless of the designation Lactobacillus paracasei S01 DSM 26760 or Lactobacillus paracasei LPC-S01 DSM 26760 adopted by the Applicant.

Said compositions of the invention for use in the methods or treatment of the invention comprise: (i) said mixture M of the invention comprising or, alternatively, consisting of Lactobacillus paracasei DG® CNCM I-1572 and/or Lactobacillus paracasei LPC-S01 DSM 26760; and, optionally, (ii) at least one food grade or pharmaceutical grade additive and/or excipient.

According to an aspect of the invention, said mixture M included in the composition of the invention may comprise or, alternatively, consist of:
Lactobacillus paracasei DG® CNCM I-1572 and/or Lactobacillus paracasei LPC-S01 DSM 26760; and
at least one further strain of bacteria selected from group (I) consisting of:
(a) Bifidobacterium breve BbIBS01 DSM 33231,
(b) Bifidobacterium breve BbIBS02 DSM 33232,
(c) Bifidobacterium animalis subsp. lactis BIIBS01 DSM 33233,
(d) Lactobacillus plantarum LpIBS01 DSM 33234,
(e) Bifidobacterium bifidum BbfIBS01 DSM 32708; and a mixture thereof.

The strain of bacteria belonging to the species Bifidobacterium breve identified as Bifidobacterium breve BbIBS01 was deposited at the Deutsche Sammlung von Mikroorganismen und Zellkulturen GmbH (DSMZ) under the deposit number DSM 33231 on 31 Jul. 2019 by SOFAR S.p.A. (in short, BbIBS01 or B. breve BbIBS01 DSM 33231).

The strain of bacteria belonging to the species Bifidobacterium breve identified as Bifidobacterium breve BbIBS02 was deposited at the Deutsche Sammlung von Mikroorganismen und Zellkulturen GmbH (DSMZ) under the deposit number DSM 33232 on 31 Jul. 2019 by SOFAR S.p.A. (in short, BbIBS02 or B. breve BbIBS01 DSM 33232).

The strain of bacteria belonging to the species Bifidobacterium animalis identified as Bifidobacterium animalis subsp. lactis BIIBS01 was deposited at the Deutsche Sammlung von Mikroorganismen und Zellkulturen GmbH (DSMZ) under the deposit number DSM 33233 on 31 Jul. 2019 by SOFAR S.p.A. (in short, BIIBS01 or B. animalis subsp. lactis BIIBS01 DSM 33233).

The strain of bacteria belonging to the species Lactobacillus plantarum identified as Lactobacillus plantarum LpIBS01 was deposited at the Deutsche Sammlung von Mikroorganismen und Zellkulturen GmbH (DSMZ) under the deposit number DSM 33234 on 31 Jul. 2019 by SOFAR S.p.A. (in short, LpIBS01 or L. plantarum LpIBS01).

The strain of bacteria belonging to the species Bifidobacterium bifidum identified as Bifidobacterium bifidum MIMBb23sg=BbflBS01, or a derivative thereof, wherein said strain of bacteria was deposited at the Deutsche Sammlung von Mikroorganismen und Zellkulturen GmbH (DSMZ) under the deposit number DSM 32708 on 4 Dec. 2017 by Sofar S.p.A. (in short, BbfIBS01 or Bifidobacterium bifidum BbfIBS01 DSM 32708). It is clarified that it is always and only the same strain of bacteria regardless of the designation Bifidobacterium bifidum BbfIBS01 DSM 32708 or Bifidobacterium bifidum MIMBb23sg DSM 32708 adopted by the Applicant.

All of the bacterial strains mentioned in the present invention (i.e. DG®, LPC-S01, BbIBS01, BbIBS02, BIIBS01, LpIBS01 and BbfIBS01) were deposited in accordance with the provisions of the Budapest Treaty. The Depositor of the strains of bacteria described and/or claimed in the present patent application and the patent application owner hereby express their consent to make all such strains available for the entire duration of the patent.

Advantageously, said strains of bacteria included in the composition of the invention (i.e. DG®, LPC-S01, BbIBS01, BbIBS02, BIIBS01, LpIBS01 and BbflBS01) are viable bacterial strains (probiotics). Alternatively, said bacterial strains of the invention may be a derivative of the viable strain as defined in the present invention.

In the context of the present invention, the term “derivative” of the bacterial strain (or “derivative” of the viable bacterial strain) is understood to mean a postbiotic or parabiotic, such as, for example, the tyndallized, sonicated, inactivated by radiation (preferably gamma radiation) bacterial strain, the lysates or homogenates of the bacterial strain, the extracts or wall fraction of the bacterial strain, the metabolites or metabolic bioproducts or exopolysaccharides (EPS) generated by the bacterial strain and/or any other derivative product of the bacterial strain known to the skilled person in the art. Said derivatives are obtained according to methodologies known to the skilled person in the art. Preferably by the term “derivative” of the bacterial strain is meant the tyndallized, sonicated, radiation-inactivated (preferably gamma radiation) strain, the lysates or homogenates of the bacterial strain, the extracts or parietal fraction of the bacterial strain; more preferably the tyndallized strain.

Examples of mixtures of bacterial strains (En) included in the mixture M, optionally together with at least one probiotic selected from group (II) and/or an additional active component selected from group (III), wherein said mixture M is included in the composition of the invention, are given below:
E1: DG® and LPC-S01;
E2: DG® and at least one strain selected from group (I);
E3: LPC-S01 and at least one strain selected from group (I);
E4: DG® and/or LPC-S01, and BbflBS01 and at least one strain selected from group (I);
E5: DG® and/or LPC-S01, and a mixture of BbIBS01, BbIBS02, BIIBS01 and LpIBS01;
E6: DG® and/or LPC-S01, BbfIBS01 and a mixture of BbIBS01, BbIBS02, BIIBS01 and LpIBS01.

The mixture M included in the composition of the invention, in addition to the strain L. paracasei DG® CNCM I-1572 and/or L paracasei LPC-S01 DSM 26760 and optionally at least one or more strains of bacteria selected from group (I) and/or at least one further active component selected from group (III) described below, may further comprise at least one prebiotic, preferably selected from group (II) consisting of: inulin, fructo-oligosaccharide (FOS), galacto-oligosaccharide (GOS), xylitol-oligosaccharide (XOS), guar gum, lactoferrin, and a mixture thereof; preferably inulin.

Advantageously, mixture M included in the composition of the invention comprises or, alternatively, consists of L. paracasei DG® CNCM I-1572 and/or L. paracasei LPC-S01 DSM 26760 and inulin; preferably L. paracasei DG® CNCM I-1572 and inulin.

Mixture M included in the composition of the invention, in addition to the L. paracasei strain DG® CNCM I-1572 and/or L. paracasei LPC-S01 DSM 26760 and optionally at least one or more bacterial strains selected from group (I) and/or at least one prebiotic selected from group (II), may further comprise at least one further active component selected from group (III) consisting of:
group A, B, C, D, E and/or K vitamins, preferably a group B vitamin and/or vitamin D;
antioxidant substances such as glutathione, polyphenols such as resveratrol and trans-resveratrol, coenzyme Q10, astaxanthin, lycopene;
plant substances (botanicals) or extracts thereof with an intestinal relaxing action, such as valerian, passion flower, lemon balm, hawthorn, camomile;
minerals or salts thereof, e.g. zinc, selenium, magnesium, potassium;
monounsaturated fatty acids, such as omega 9, and/or polyunsaturated fatty acids, such as omega 3 and omega 6;
immunostimulant substances, anti-diarrhoeal substances, and/or nutrients; and and a mixture thereof.

Advantageously, mixture M included in the composition of the invention comprises or, alternatively, consists of L. paracasei DG® CNCM I-1572 and/or L. paracasei LPC-S01 DSM 26760 and group B vitamins and vitamin D; preferably L. paracasei DG® CNCM I-1572 and group B vitamins and vitamin D.

In the context of the present invention, food grade or pharmaceutical grade acceptable “additives and/or excipients”, optionally included in the composition of the invention together with said strains of bacteria, comprise all auxiliary substances known to the person skilled in the art for the preparation of compositions in solid form, semi-solid or liquid form, such as, for example, carriers, diluents, solvents, solubilisers, acidifiers, thickeners, sweeteners, flavourings, colourings, sweeteners, lubricants, surfactants, preservatives, stabilisers, pH stabilising buffers, and mixtures thereof. For example, in the case of compositions in drops form for oral administration to newborns or subjects from 1 to 12 months, seed oil may be used as a diluent.

Said mixtures M or compositions of the invention, according to any one of the described embodiments, may be a pharmaceutical composition (or Live Biotherapeutic Products), a composition for a medical device, a composition for a food supplement, a food (or novel food or food for special medical purposes (FSMP)), a composition for a food supplement or food.

The bacterial strains, mixtures and compositions thereof of the present invention, according to any one of the described embodiments, may be formulated for oral or nasal administration, preferably for oral use, in a solid form or, alternatively, in a liquid form, e.g. in the form aqueous or oil (e.g., various seed oils or sunflower seed oil) based drops.

Advantageously, said at least one bacterial strain or each bacterial strain is present in the mixture M or in the composition of the invention, with respect to a daily dose of the M-mixture or composition of the invention, in a concentration in the range from 10×10⁶ CFU to 10×10¹² CFU, preferably from 10×10⁸ CFU to 10×10¹⁰ CFU, more preferably in a concentration of about 10×10⁸ CFU or 10×10⁹ CFU (CFU: Colony Forming Unit).

For example, a daily dose of a mixture or composition of the invention (e.g. drops for oral administration) comprises: ≥1×10⁹ live bacteria of L. paracasei DG® CNCM I-1572, and seed oil.

The above daily dosages may be administered to the subject in need thereof in a individual dose (single dose) or in repeated doses, e.g. two, three or four daily doses. For example, 15-5 drops (oil-based), preferably from 12 to 8 drops, e.g. about 9-10 drops, of the composition of the invention twice a day equal to about 2 billion of strains of bacteria, preferably L. paracasei DG® CNCM I-1572.

In order to assess the number of live bacterial strains in the compositions or mixtures M of the present invention, these compositions or mixtures M can be analysed by plate count method to determine the CFU value.

For the sake of clarity, in order to achieve the purpose of the present invention, the strain(s) of bacteria, the prebiotics and/or further (optional) active components included in the mixture (M) of the invention may also be administered separately (preferably in a time interval from 30 minutes to 60 minutes) and in any order but, preferably, are administered to a subject at the same time, even more preferably in a single composition to achieve a faster effect and for ease of administration. When said strains and (optional) active components are administered in a single composition, said single composition corresponds to the composition of the present invention.

Unless otherwise specified, the expression composition or mixture or other comprising a component in an amount “within a range from x to y” means that said component may be present in the composition or other in all amounts present in said range, even if not explicitly stated, extremes of the range included.

Unless otherwise specified, a statement that a composition ‘comprises’ one or more components or substances means that other components or substances may be present in addition to that, or those, specifically indicated.

“Method of treatment” in the context of the present invention means an intervention on a subject in need thereof, comprising the administration of the strain of bacteria or a composition of the invention to the subject in a therapeutically effective amount, having as its purpose the elimination, reduction/decrease or prevention of a pathology or disease and related symptoms or disorders.

The term “subject(s)” in the context of the present invention refers to mammals (animals and humans), preferably human subjects of neonatal age (from 0 to 4 weeks of age) and/or from 1 month to ≤12 months of life (from the beginning of the 2nd month until before the end of the 12th month).

Advantageously, subjects treated or administered with the strains or mixtures or compositions of the invention may be newborn subjects or from 1 month to ≤12 months of life born by caesarean section and/or born preterm.

The term ‘therapeutically effective amount’ refers to the amount of active compound and/or strain of bacteria that elicits the biological or medicinal response in a tissue, system, mammal or human being that is sought and defined by an individual, researcher, veterinarian, physician or other clinician or health care worker.

The term “medical device” in the context of the present invention is used in the meaning according to Legislative Decree No. 46 of 24 Feb. 1997 or according to the new Medical Device Regulation (EU) 2017/745 (MDR).

The term ‘novel food’ in the context of the present invention is used in the meaning according to EC Regulation 258 of 1997.

### EMBODIMENTS

Preferred embodiments FRn of the present invention are as follows.

FR1. A strain of bacteria or a composition for use in a newborn subject, in the first 4 weeks of life, and/or in a subject from 1 month to ≤12 months in treatment, preventive and/or curative, of a subject having gastro-intestinal tract disorders of an inflammatory and/or functional nature, such disorders being selected from the group comprising or, alternatively, consisting of: neonatal colic, intestinal colic, chronic enterocolitis or chronic enterocolitis in preterm newborns, necrotizing enterocolitis, sepsis, irritable bowel syndrome (IBS), IBS with prevalent constipation or constipated bowel, IBS with prevalent diarrhoea or diarrhoeic bowel, IBS with alternating bowel, unclassified IBS, chronic inflammatory bowel disease (IBD), Crohn's disease, ulcerative rectocolitis, asthma, obesity, type 1 diabetes, atopic dermatitis, multiple sclerosis, cancer, autism, allergies, immune-mediated, autoimmune disorders, selected from coeliac disease, Graves' disease, rheumatoid arthritis, Hashimoto's thyroiditis, diabetes mellitus type 1, systemic lupus erythematosus (lupus), vasculitis, Addison's disease, polymyositis, Sjögren's syndrome, progressive systemic sclerosis, glomerulonephritis (inflammation of the kidneys), infertility and support of body weight gain in said subject;
wherein said strain of bacteria belongs to the species Lactobacillus paracasei;
wherein said composition comprises:
(i) a mixture M comprising or, alternatively, consisting of at least one strain belonging to the species Lactobacillus paracasei; and
(ii) at least one food-grade or pharmacological grade additive and/or excipient.

FR2. The strain for use or the composition for use according to FR1,
wherein said strain of bacteria is selected from Lactobacillus paracasei DG® CNCM I-1572, Lactobacillus paracasei LPC-S01 DSM 26760, and a mixture thereof;
wherein said composition comprises:
(i) a mixture M comprising or, alternatively, consisting of: Lactobacillus paracasei DG® CNCM I-1572 and/or Lactobacillus paracasei LPC-S01 DSM 26760; and
(ii) at least one food-grade or pharmacological grade additive and/or excipient.

FR3. The strain for use or the composition for use according to FR1 or FR2, wherein said strain or composition is for use in a newborn subject and/or a subject from 1 month to <12 months in the treatment, preventive and/or curative, of neonatal or intestinal colic, or chronic or chronic enterocolitis in preterm newborns.

FR4. The strain for use or the composition for use according to FR1 or FR2, wherein said strain or composition is for use in a newborn subject and/or a subject from 1 month to ≤12 months in the treatment, preventive and/or curative, of gastro-intestinal infections by pathogenic micro-organisms or gastro-intestinal infections by parasites.

FR5. The strain for use or the composition for use according to FR1 or FR2, wherein said strain or composition is for use in a newborn subject and/or a subject from 1 month to ≤12 months in the treatment, preventive and/or curative, of allergies.

FR6. The strain for use or the composition for use according to FR1 or FR2, wherein said strain or composition is for use in a newborn subject and/or a subject from 1 month to ≤12 months in the treatment, preventive and/or curative, of immune-mediated, autoimmune disorders, such as coeliac disease Graves' disease, rheumatoid arthritis, Hashimoto's thyroiditis, type 1 diabetes mellitus, systemic lupus erythematosus (lupus), vasculitis, Addison's disease, polymyositis, Sjögren's syndrome, progressive systemic sclerosis, glomerulonephritis (inflammation of the kidneys) and infertility.

FR7. The strain for use or the composition for use according to FR1 or FR2, wherein said strain or composition is for use in a newborn subject and/or a subject from 1 month to ≤12 months in the treatment, preventive and/or curative, of supporting the body weight gain of the subject.

FR8. The composition for use according to any one of FR1-7, wherein said (i) mixture M comprises or, alternatively, consists of:
Lactobacillus paracasei DG® CNCM I-1572 and/or Lactobacillus paracasei LPC-S01 DSM 26760; and
at least one further strain of bacteria selected from the group consisting of:
   Bifidobacterium breve BbIBS01 DSM 33231,
   Bifidobacterium breve BbIBS02 DSM 33232,
   Bifidobacterium animalis subsp. lactis BIIBS01 DSM 33233,
   Lactobacillus plantarum LpIBS01 DSM 33234,
   Bifidobacterium bifidum BbfIBS01 DSM 32708; and
      a mixture thereof.

FR9. The composition for use according to any one of FR1-8, wherein said (i) mixture M further comprises at least one further active component selected from:
at least one vitamin from the group A, B, C, D, E and/or K, preferably a group B vitamin and/or vitamin D;
antioxidant substances such as glutathione, polyphenols such as resveratrol and trans-resveratrol, coenzyme Q10, astaxanthin, lycopene;
plant substances, botanicals, or extracts thereof with an intestinal relaxing action, preferably selected from valerian, passion flower, lemon balm, hawthorn and camomile;
minerals or salts thereof, e.g. zinc, selenium, magnesium, potassium;
monounsaturated fatty acids, such as omega 9, and/or polyunsaturated fatty acids, such as omega 3 and omega 6;
immunostimulant substances, anti-diarrhoeal substances, and/or nutrients;
at least one prebiotic, preferably selected from group (II) consisting of: inulin, fructo-oligosaccharide (FOS), galacto-oligosaccharide (GOS), xylitol-oligosaccharide (XOS), guar gum, lactoferrin, and a mixture thereof; more preferably inulin; and
   and a mixture thereof

FR10. The strain per use or the composition per use according to any one of FR1-9,
wherein said strains are viable bacterial strains or derivatives of bacterial strains, wherein said derivatives of bacterial strains are selected from: tyndallized, sonicated, radiation-inactivated (preferably gamma radiation) bacterial strain, lysate or homogenised bacterial strain, extract or wall fraction of bacterial strain; preferably tyndallized bacterial strain.

### Experimental Part

The Applicant carried out a clinical study in newborns in order to evaluate the effects on the gut microbiota of the host in the first 3 months of life following the administration of the Lactobacillus paracasei DG strain® CNCM I-1572 probiotic (viable cells).

### 1. Objectives of the Clinical Study

### Primary Objective of the Study:

To assess whether supplementation of a composition according to the invention in drops form (oil-based) for oral administration comprising the L. paracasei strain CNCM I-1572 (in short, composition under analysis) for 28 days changes the composition of the faecal gut microbiota in terms of the concentration of Lactobacillus paracasei CNCM I-1572 in the study population.

### Composition Under Analysis:

Sunflower seed oil (Heliantus annuus L., DL-alpha-tocopherol, Lactobacillus paracasei CNCM I-1572 (not less than 14 billion of live cells per 8 ml).

### Secondary Objectives of the Study:

To assess the intake of the composition under analysis for 28 days in terms of:
Change of the composition (real-time PCR) of the faecal gut microbiota with prediction of the ecology of the microbiota system at 56 and 84 days after intake;
Change of the functional activity of the faecal gut microbiota (metabolomics) with prediction of the ecology of the microbiota system at 28, 56 and 84 days after intake;
Change in stool amount and quality (frequency and consistency);
Occurrence of neonatal colic defined according to Rome IV criteria;
Safety and tolerability of the product;
Patient growth.

### 2. Organisation of the Study

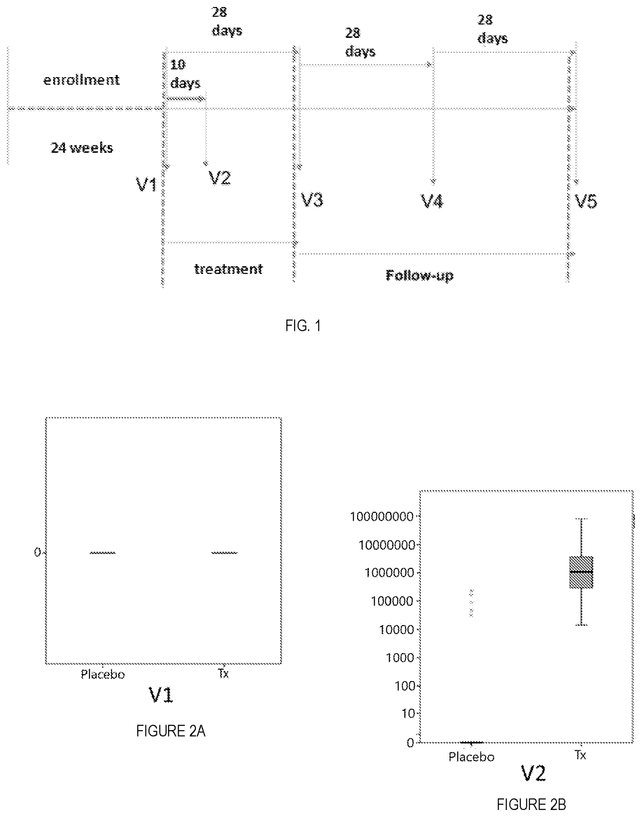

### Timing of the Search (FIG. 1)

Enrolment period: 24 weeks;
Treatment duration: 4 weeks;
Follow-up duration: 8 weeks;
Study per patient duration: 12 weeks (84 days);
Total study duration: 36 weeks.

### 3. Study Design

Randomised, placebo-controlled, double-blind, single-centre clinical study. Newborns were stratified by birth weight into three groups:
Normal Birth Weight (>2500 g, NBW),
Low Birth Weight (1500-2500 g LBW),
Very Low Birth Weight (1000-1500 g, VLBW).

The trial was conducted at the U.O. of Neonatology of the Policlinico Casilino in Rome as the only participating and coordinating centre.

### 3.1. Study Population

The study involves the enrolment of 60 preterm and term newborns, divided as follows: 20 NBW, 20 LBW, 20 VLBW.

### 3.2 Types of Intervention

The 60 subjects enrolled were randomised in a 1:1 ratio into each of the three groups in two arms:

### Arm 1:

Test composition, in drops form, 9 drops twice a day, equal to 2 billion CFU (colony-forming units) of L. paracasei DG® (CNCM I-1572) for 4 weeks (28 days).

### Arm 2:

CONTROL: Placebo, 9 drops twice a day for 4 weeks (28 days).

### 3.3 Duration of the Study

A 12-week period allows to evaluate the changes in the gut microbiota based on the persistence of the L. casei DG strain® (Lactobacillus paracasei CNCM I-1572) determined by the intake of the composition under analysis. The 12-week period is divided into 4 weeks of treatment and 8 weeks of follow-up. Including an enrolment period of approximately 24 weeks, the total duration of the study will be 36 weeks.

### 4. Selection of Study Subjects

### 4.1 Inclusion Criteria

0-48 hours of life;
Newborns of both sexes with a birth weight ≥1000 g;
Newborns by caesarean section.

### 4.2 Exclusion Criteria (Including Only the Main Ones)

Vaginal delivery;
Extremely low birth weight (ELBW) (<1000 g);
Established or suspected systemic infections;
Any severe pathology that in the opinion of the investigator may interfere with the treatment;
Intake of probiotics other than the trial product by the newborn or breastfeeding mother since the start of the study.
Systemic antibiotic therapy or prophylaxis from birth and for the entire duration of the study (NBW only);
Therapy or prophylaxis with systemic antibiotics by the breastfeeding mother (of NBW newborn) in the 30 days prior to birth and for the entire duration of the study.

The newborn will be considered eligible for the Per Protocol population if he/she has taken at least 80% and no more than 120% of the prescribed treatment.

### 5. Procedures and Methods

Control visits with contextual collection of a stool sample, if possible:

V1 day 1 (0-48 hours of life); V2 day 10 (+/−2 days); V3 day 28 (+/−3 days); V4 day 56 (+/−3 days); V5 day 84 (+/−3 days) (FIG. 1).

Furthermore, during the study, faecal samples collected from parents were delivered according to the following scheme (1 sample collected within 24 hours before each visit):

### Pre-Treatment:

Faeces in the first 48 hours of life and, when available, meconium.

### In the Treatment Phase:

Faeces 10 days after starting the intake;
Faeces after 28 days from the start of intake;

### In the Follow-Up Phase:

Faeces after 56 days from the start of intake;
Faeces after 84 days from the start of intake;

### Chemical-Physical Analysis of Faecal Samples:

real-time PCR analysis to assess the bacterial count of L. casei strain DG® (Lactobacillus paracasei CNCM I-1572);
Metagenomics and metabolomics analyses to characterise the gut microbiota pre- and post-administration of the probiotic.

Specifically, the microbiota was analysed through nucleotide sequence analysis of portions of the gene coding for the bacterial ribosomal subunit 16S rRNA. Specifically, a metagenomic strategy consisting of the following steps will be adopted:
Extraction, quantification and normalisation of metagenomic DNA from faecal samples;
PCR amplification of the V3-V4 hypervariable regions of the bacterial gene coding for 16S rRNA;
Quantification of PCR products;
Sequencing with Illumina MiSeq technique;
Bioinformatic sequence analysis (characterisation of microbial communities, hierarchical clustering, taxonomic analysis, construction of phylogenetic dendrograms with heatmaps).

For metabolomic analysis, metabolites of microbial origin produced as volatile organic compounds (VOCs) were identified and quantified. These compounds were extracted using a GC-MS/SPME (Gas Chromatography coupled to Mass Spectrometry by Solid Phase Micro Extraction) system. For VOCs extraction, a carboxy-polydimethylsiloxane (CAR-PDMS) coated fibre (85 μm) was used in the SPME process. From each sample, analysed in triplicate, an average of 100-500 mg was placed in 10 ml glass vials with the addition of 4-methyl-2-pentanol as an internal standard (IS). Subsequently, faecal samples were equilibrated for 10 minutes at 45° C. The fibre was exposed to each sample for 45 minutes before injection into GC-MS (Hewlett Packard 6890 GC) coupled to a 5973C selective mass detector and equipped with a Supelcowax 10 capillary column. Metabolites are identified using their retention times (Rt) relative to the pure compound. Chromatograms are integrated and identified by comparing the fragment pattern with those from the NIST library followed by manual visual inspection. Quantitative metabolite data are obtained by interpolation of the relative areas with respect to the IS area.

### 6. Effectiveness and Safety Evaluation

### 6.1. Evaluation of Effectiveness

### Primary Endpoint

Bacterial genome count of L. casei DG® at day 28 by Real Time PCR.

### Secondary Endpoints

Indicators of dysbiosis of the gut microbiota (α-diversity and β-diversity) at day 28, 56 and 84;
Bacterial L. casei DG® genome count at days 56 and 84;
Number, consistency, amount and colour of faecal evacuations on the day before the visits (day 1, 10, 28, 56, 84) using the Amsterdam stool scale for newborns;
Chemico-physical analysis of faeces (day 1, 10, 28, 56, 84);
Quantitative assessment of the volatilome, i.e. volatile faecal metabolites (chemical categories: short chain fatty acids (SCFA), alcohols, ketones, aldehydes, thiols, acids, esters, pyrazines, pyridines, phenols, furans, terpenes, alkanes, alkenes, etc.), by assessing the change in the overall metabolic profile of the microbiota at day 28, 56, 84;
Assessment of the presence of colic according to ‘Rome IV’ diagnostic criteria;
Evaluation of adverse events.

### 7. Statistical Analysis

### Statistical Methods

Categorical data are represented in terms of counts and percentages, while continuous data as averages and standard deviations or medians and ranges.

Comparisons of categorical data are made by the Chi-square test or Fisher's exact test, as appropriate.

Continuous data are compared by Student's t-test (for independent or dependent data), Anova and Anova for repeated measures, where the appropriate statistical tests determined the normality of the data; in the case of data not normally distributed, non-parametric tests such as sum rank test (Mann Whitney Rank sum test) or sum signed rank test (Wilkoxon Signed Rank), Kruskal Wallis test or Friedman's test are used. Multivariate regression techniques are used if indicated to assess the simultaneous effect of several independent variables on a response variable, or to identify possible confounders and/or analyses for temporal data. Results are considered statistically significant with a P-value<0.05.

All analyses are performed with Stata 15 software (StatCorp).

### 8. Microbiological Results of the Study

8.1 Primary endpoint: bacterial genome count of L. casei DG® at day 28 by Real Time PCR

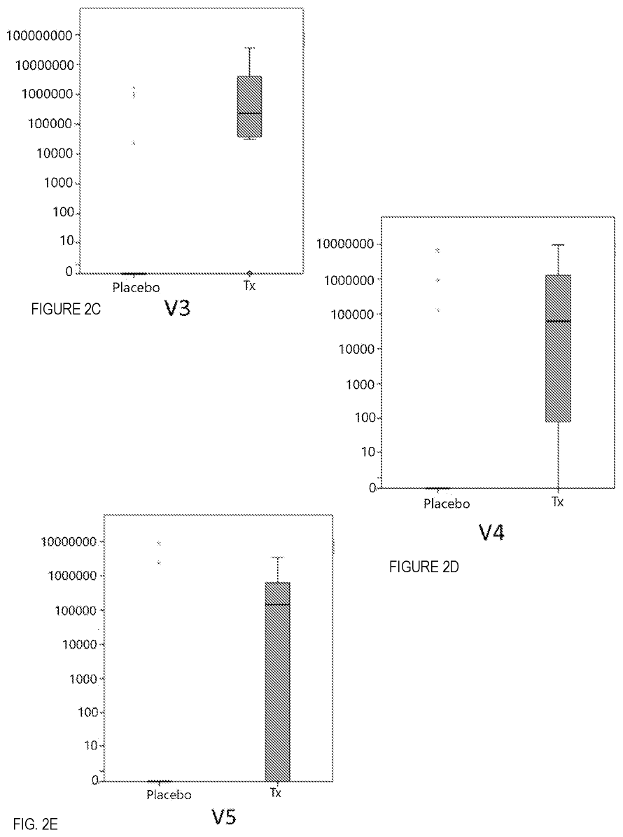

The results of the faecal samples collected during the V1-V5 control visits are schematised in the diagrams in FIGS. 2A to 2E.

Such diagrams (box plot) show the concentrations of L. casei DG® in the placebo group and in the ‘Tx’ group to which the composition under analysis was administered, for each follow-up point. For each group, the median, 25th percentile, 75th percentile, minimum and maximum value of the L casei DG® concentration (CFU/ml) are shown.

The probiotic L. casei DG®, detected in the intestines of subjects in the treatment group starting 10 days after the first administration, was persistent up to 84 days after intake, with a slight decrease starting at point V3 of the follow-up (56th day of treatment).

### 8.2 Metagenomic Analysis

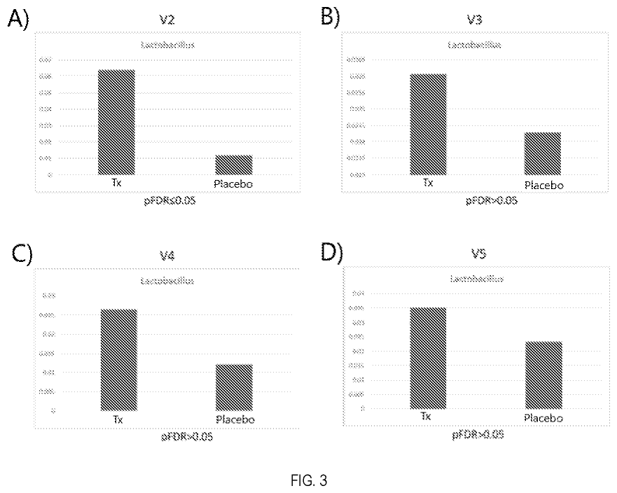

FIGS. 3A to 3D show histograms of the relative abundance of Lactobacillus spp. at the time points of control visits V2-V5.

From the analysis of the ecology of the microbiota, it can be deduced that the administration of the composition under analysis (Tx group) causes a statistically significant increase in the relative abundance of the genus Lactobacillus spp. up to ten days (point V2) after the start of administration (pFDR≤0.05) and that this increase continues to have a trend with p<0.05 at time points V4 and V5.

### 8.3 Metabolomic Analysis

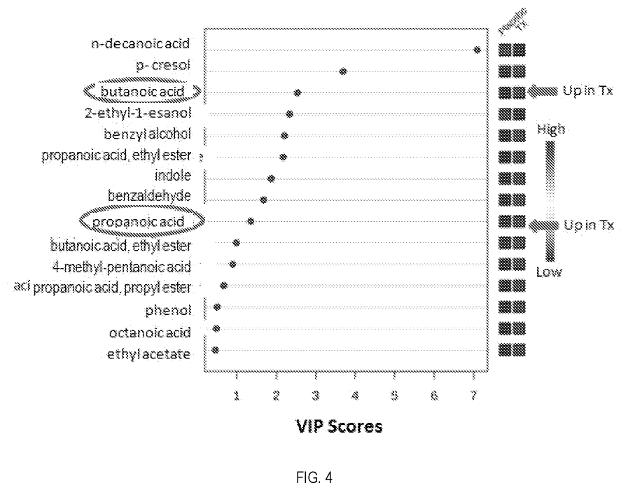

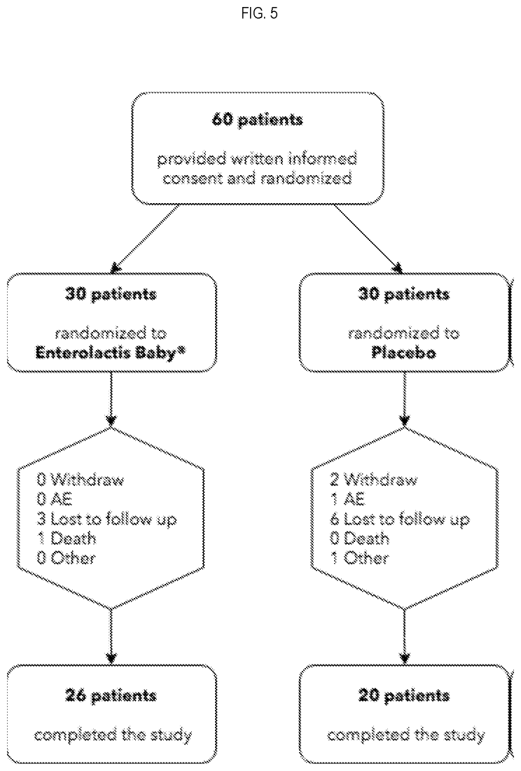

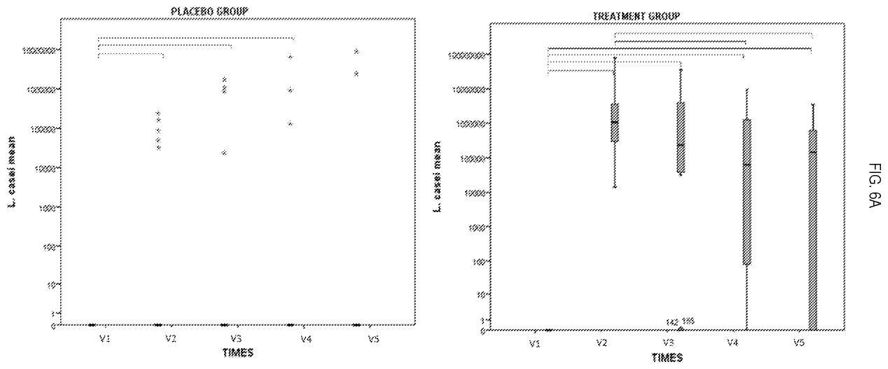

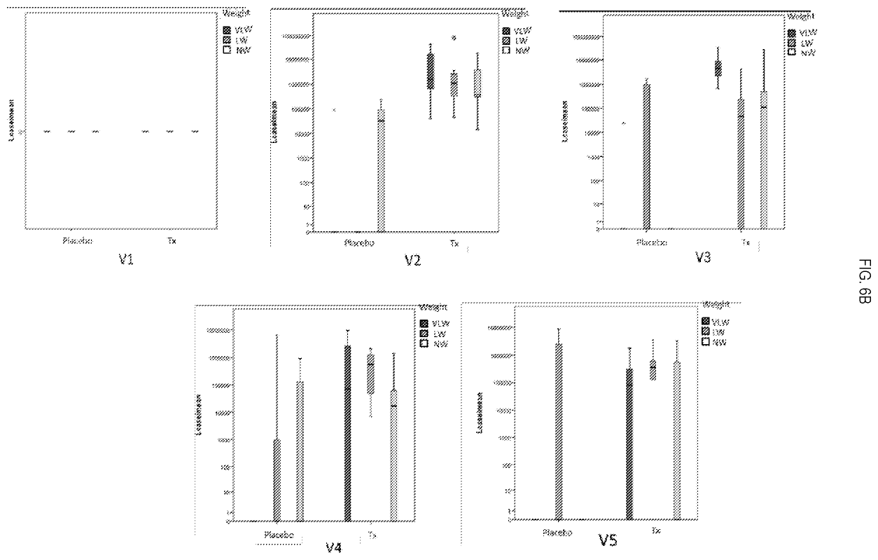

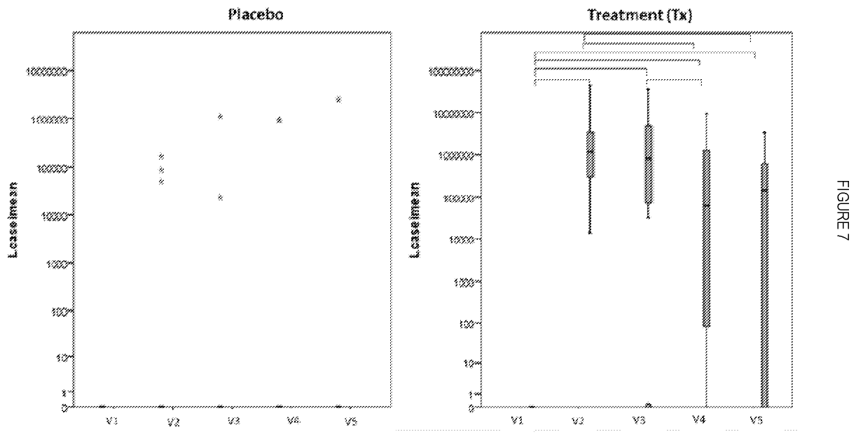

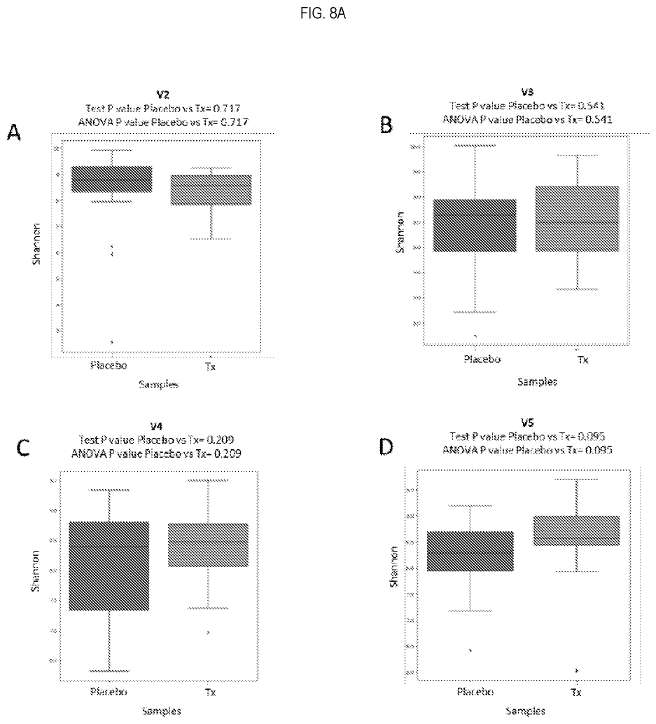

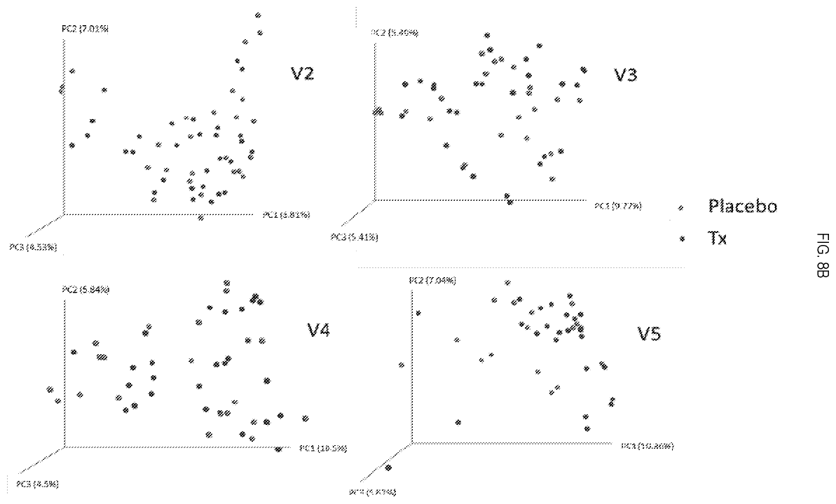

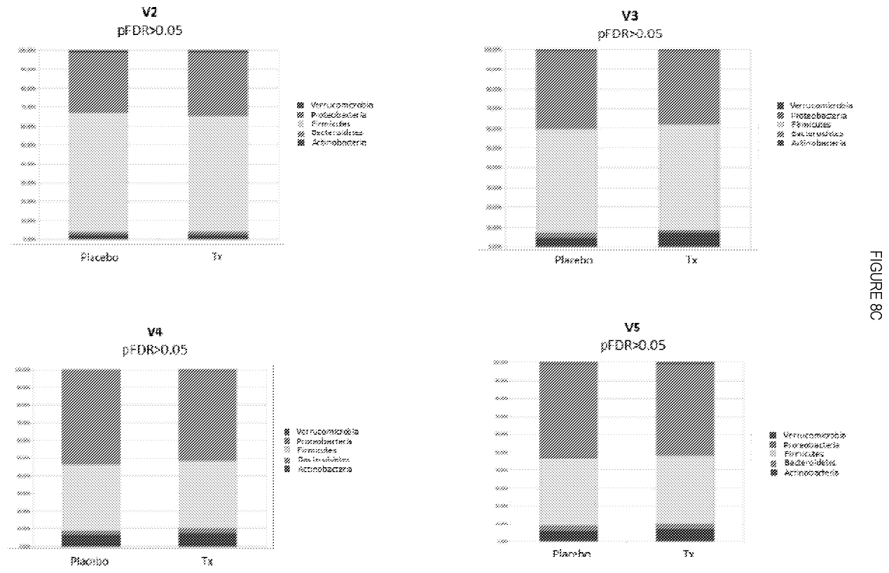

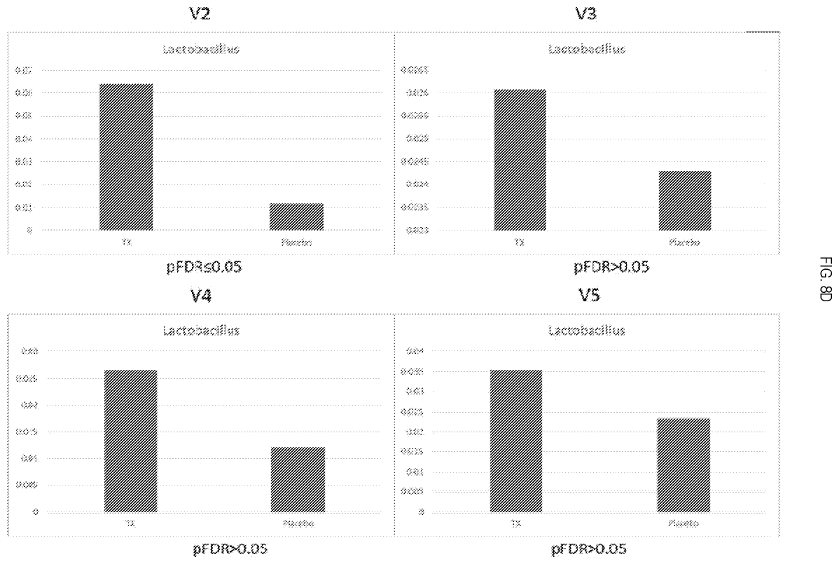

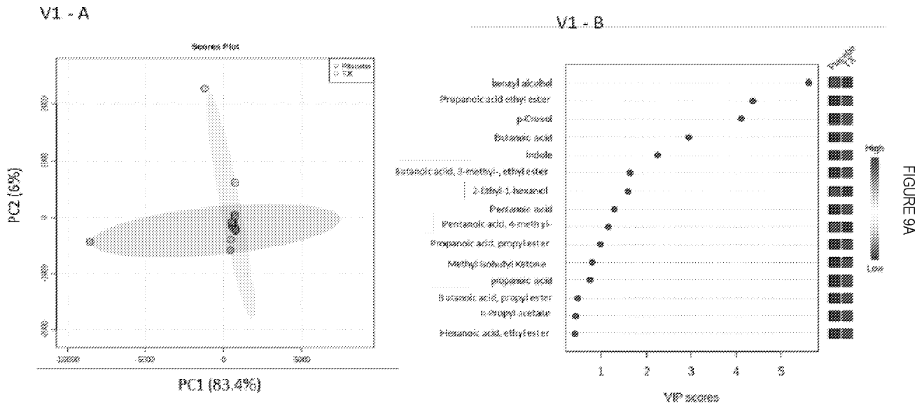

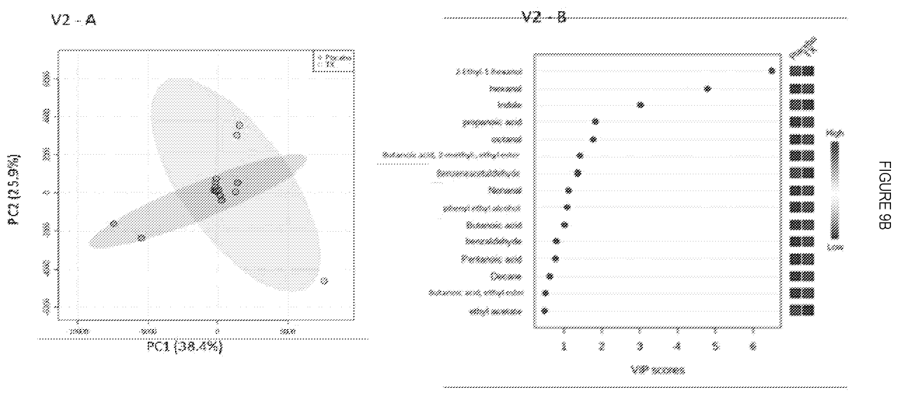

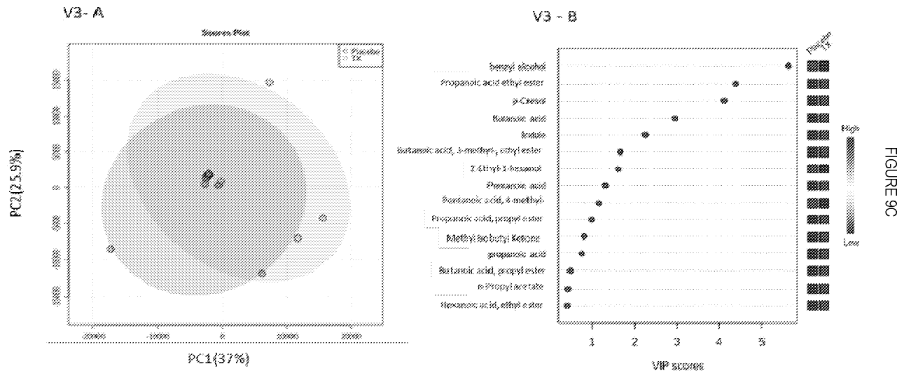

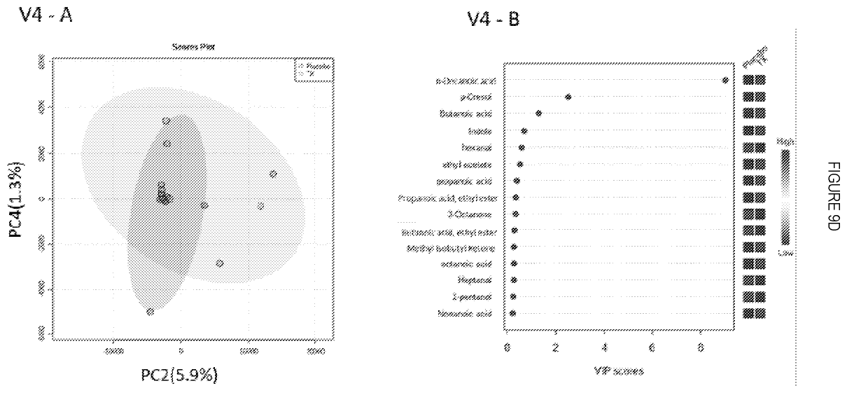

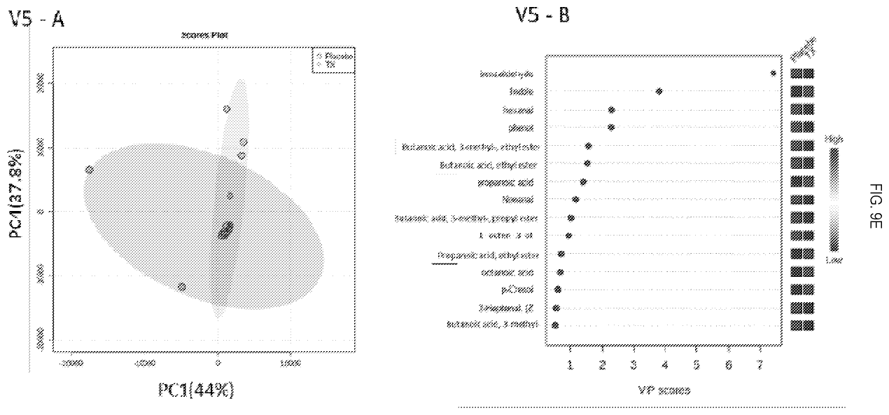

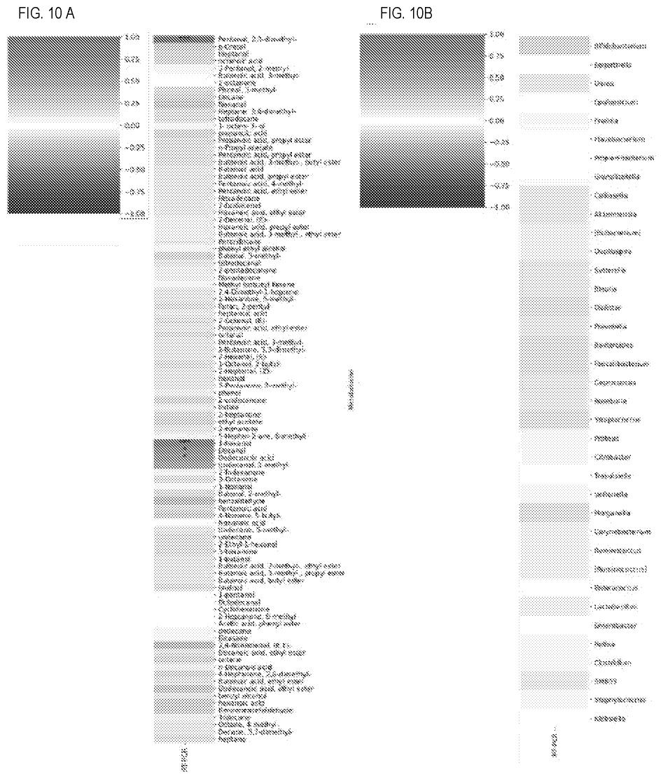

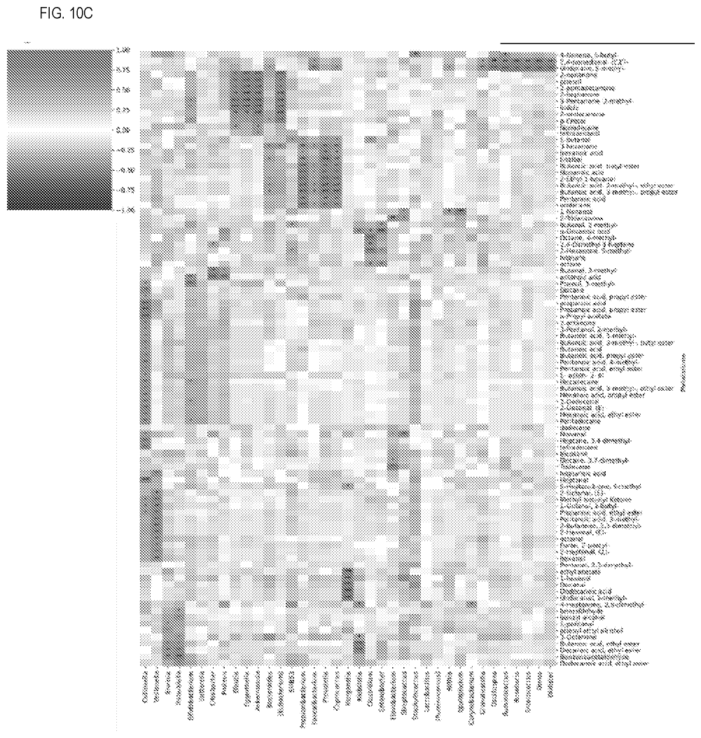

FIG. 4 shows a diagram of the results of the PLS-DA metabolomic analysis: the vertical axis lists the identified metabolites, and the horizontal axis shows the variable importance in projection (VIP).

PLS-DA analysis identified butanoic acid and propanoic acid metabolites (SCFAs) as the main players in the clustering between the placebo group and the Tx group with an increase in the concentration of said metabolites in the Tx samples.

Considering the time points separately by PLS-DA, it can be seen that the greatest separation of samples occurs between time V2 and V4 with an increase in the concentration of SCFAs in the TX samples.

Such trend is in agreement with the higher relative abundance of lactobacilli (section 8.2) in subjects in the Tx group who were administered the composition under analysis.

### 8.4 Correlations Between all Microbiological Data Obtained

The results obtained from the three different approaches discussed in the previous paragraphs (Real Time PCR, metagenomic analysis, metabolomic analysis) were correlated with each other by means of a Spearman correlation.

For the correlation analyses, the time points V2 and V3, corresponding to the administration of the composition under analysis (V2: 10 days; V3: 28 days) were selected as metagenomic, metabolomic and Real Time PCR analyses showed the most statistically significant differences between the placebo group and the Tx group treated with the composition under analysis.

By integrating the data obtained from the three approaches, it can be shown that the administration of the probiotic L. casei DG® has an effect on both the ecology and the function of the microbiota, modifying the ecological niches of the bacteria residing in the gut and the corresponding functional profile.

In particular, certain short chain fatty acids (SCFA)—such as butanoic acid and propanoic acid-showed a positive correlation with certain beneficial bacteria of the genus Faecalibactarium, Oscillospira, Eggerthella, Bacteroides, and Bifidobacterium.

### 9. Clinical Results of the Study: Stool Number, Consistency, Amount and Colour

The number of evacuations observed at each study visit was substantially higher in subjects treated with the composition under analysis than in subjects treated with placebo.

With regard to the amount of evacuations, there were no statistically significant differences between the two study groups after 10, 56 and 84 days after the start of treatment (P=0.12; 0.61 and 0.25; respectively).

Only at the end of the treatment period (day 28, V3) was observed a statistically significant difference in the amount of evacuations in favour of the subjects treated with the composition under analysis (P=0.03).

With regard to the colour and consistency of the stools (parameters assessed according to the “Amsterdam Infant stool scale”), a statistically significant difference was observed in favour of the subjects treated with the composition under analysis after 10 days (P<0.0001) and 56 days (P=0.007) after the start of treatment with regard to stool colour, and after 10 days (P=0.0016), 56 days (P<0.0001) and 84 days (P=0.05) with regard to stool consistency.

### 10. Safety Results of the Study

The results of the safety analysis are summarised in Table 1 below.

TABLE 1

| | | Treatment arm | | | |
|---|---|---|---|---|---|
| | | Placebo (N = 29) | | Tx (N = 30) | |
| Number of patients with at least one AE* | N (%) | 15 | (51.7) | 12 | (40.0) |
| Number of patients with at least one product- | N (%) | 0 | (0.0) | 0 | (0.0) |
| related AE*. | | | | | |
| Number of patients with at least one serious AE* | N (%) | 8 | (27.6) | 4 | (13.3) |
| Number of patients with at least one severe AE* | N (%) | 2 | (6.9) | 2 | (6.7) |
| Number of patients who terminated the study in | N (%) | 1 | (3.4) | 0 | (0.0) |
| advance due to an AE* | | | | | |

| | | | |
|---|---|---|---|
| Number of AE* | N | 21 | 16 |
| Number of serious AE*. | N | 10 | 6 |
| Number of related AEs* | N | 0 | 0 |
| Number of severe AEs* | N | 2 | 4 |
| Number of deaths | N | 0 | 1 |

*AE = adverse event

A total of 37 adverse events (AEs) have been recorded, of which 21 in the placebo-treated group and 16 in the group of subjects treated with the composition under analysis (Tx).

27 of the 59 subjects enrolled and randomised presented one or more adverse events (15 subjects in the placebo group; 12 subjects in the Tx group).

Only two serious adverse events (necrotising colitis and suspected sepsis) were reported in one subject treated with the composition under analysis. However, none of the serious adverse events appeared to be related to the administration of the composition under analysis.

The same judgement can be made about the serious adverse event (mild gastro-oesophageal reflux that required hospitalisation) observed in the placebo group.

The death recorded in the patient treated with the composition under analysis, caused by disseminated intravascular coagulation and septic shock, was classified by the investigator as ‘unlikely related’ to the study treatment.

All remaining severe and non-severe adverse events were considered either unrelated (N=29) or unlikely related (N=3) to the study treatment administered (placebo or composition under analysis).

No eczema or skin diseases has been recorded in the two groups during the study period.

### 11. CONCLUSIONS

In light of the above, it is concluded that:
with regard to the primary endpoint, in the Tx group treated with the composition under analysis the probiotic L. casei DG® was detected in the gut from day 10 after the start of treatment (V2; FIG. 2B), and was persistent until the end of the observation period V5 (FIG. 2E);
in relation to the relative abundance of Lactobacillus spp, the composition under analysis showed to produce a statistically significant increase in the relative abundance of lactobacilli until the end of the observation period V5 (FIG. 3D);
with regard to the PLS-DA metabolomic analysis, butanoic acid and propanoic acid metabolites were identified, which showed a higher concentration in the Tx samples (FIG. 4);
from a correlation of previous microbiological analyses, it can be deduced that the presence of butanoic acid and propanoic acid showed a positive correlation with certain species of bacteria beneficial to the microbiota of the treated subjects;
subjects treated with the composition under analysis had a statistically significant higher amount of evacuations, and better stool colour and consistency-assessed according to the Amsterdam stool scale for newborns—than the placebo-treated group;
finally, on the basis of the safety and tolerability profile analysed above, as well as the assessment of severe adverse events recorded in both study groups, the composition under analysis proved to be safe and well tolerated with a safety profile comparable to that of placebo.

The results obtained are far from obvious, since, as mentioned above, in the first weeks of life mammals, and in particular humans, cannot even be equated with children, as they have not yet completed the maturation of their internal organs. It was therefore necessary to conduct the clinical trials described above to ascertain the efficacy and safety of the strains here described, because it was by no means foreseeable that these strains would be able to provide the pharmacological response obtained, nor that they would be well-tolerated by newborns.

### Experimental Part

So far, no studies have been conducted to assess the effects of LCDG on a vulnerable population such as healthy newborns (in preterm and term newborns).

The aim of the present double-blind, placebo-controlled study was to confirm the ability of a paediatric formulation of LCDG to pass alive through the gastrointestinal tract of newborns (stratified into three groups according to birth weight: Normal birth weight, Low birth weight, Very low birth weight) during and after the administration period, by assessing its ability to positively influence the microbiota composition. The safety of the product (also monitoring weight, length and head circumference parameters), defecation frequency, stool consistency and occurence of colic were also evaluated.

### Materials and Methods

### Experimental Product

Lactobacillus paracasei, (L. casei DG®-CNCM I-1572; LCDG) was supplied by SOFAR S.p.A. 9 drops of Lactobacillus paracasei, corresponding to 1 billion colony-forming units (CFU) of LCDG, were administered twice a day for 28 days. The drops were administered directly on the tongue or mixed with a cold or lukewarm liquid.

### Subjects of the Study

This study was conducted from September 2018 to March 2021. Patients participating in the study were selected from a group of preterm and term newborns, at the Hospital Neonatology Unit, Policlinico Casilino, Rome-Italy. Inclusion criteria for the study were determined as follows: age between 0 and 48 hours, newborns of both sexes with birth weight ≥1000 g, newborns delivered by caesarean section and written informed consent from parents/guardians of the newborns. Exclusion criteria were: vaginal delivery, extremely low birth weight (ELBW) (<1000 g), established or suspected systemic infection, known severe neurological disease, known severe metabolic disease, known genetic disease and chromosomal disorders, severe malformations (e.g. short bowel syndrome; bowel obstruction; ductus arteriosus patency may be included if asymptomatic and does not require treatment), known severe primary or secondary maternal immunodeficiency, known maternal food allergies, maternal diabetes (including gestational diabetes), recent or suspected history of alcohol or drug abuse by the mother, any severe condition that the investigator believes may interfere with treatment, inadequate reliability or presence of conditions that could result in non-compliance or non-observance of the protocol by the patient. In addition, newborns were prohibited from receiving probiotics other than the trial product for the entire duration of the study, and breastfeeding mothers were prohibited from receiving any probiotic for the entire duration of the study. Only for normal body weight (NBW) newborns, systemic antibiotic treatment or prophylaxis was not allowed from birth and for the entire duration of the study, and systemic antibiotic treatment or prophylaxis was not allowed by a breastfeeding mother (of an NBW infant) in the 30 days prior to the study and for the entire duration of the study.

Each parent/guardian of the newborn signed an informed consent to participate in the study and received an explanatory sheet with the conditions of use of the trial product. Based on the inclusion/exclusion criteria, 60 preterm and term newborns were selected.

### Study Design

The aim of this 12-week, randomised, double-blind, single-centre, placebo-controlled clinical trial was to evaluate whether the administration of LCDG to newborns in the first 48 hours after delivery changes the composition of the faecal gut microbiota after product supplementation for 28 days. The 12-week period was divided into 4 weeks of treatment and 8 weeks of follow-up.

The children were involved in 5 on-site visits: Visit 1 (V1; 0-48 hours after birth), Visit 2 (V2; 10 days+/−2 days), Visit 3 (V3; 28 days+/−3 days; end of product intake), Visit 4 (V4; 56 days+/−3 days) and Visit 5 (V5; 84 days+/−3; end of study).

At V1, newborns will be stratified into three groups according to birth weight: Normal birth weight (>2500 g, NBW), low birth weight (1500-2500 g LBW), very low birth weight (1000-1500 g, VLBW).

Eligible patients thus stratified will be randomised at a ratio of 1:1 into one of the following arms: Arm 1: Treatment group: 9 drops twice a day of LCDG corresponding to 2 billion CFU (colony-forming units) for 4 weeks (28 days) starting at 48 hours of life; Arm 2: Placebo group: 9 drops twice a day of placebo (product indistinguishable from the test product) for 4 weeks (28 days) starting at 48 hours of life.

During the same visit, the demographic data of the baby and the mother and a stool sample from the child were collected. The primary outcome was the composition of the faecal gut microbiota in terms of bacterial genome counts for LCDG at day 28th (V3) of treatment by Real Time PCR. The secondary outcomes assessed during the study were: modification of the functional activity of the faecal gut microbiota (metabolomics analysis-α- and β-diversity) with prediction of the microbiota ecology at 28, 56 and 84 days (V3,V4 and V5 respectively) after the start of administration; change in the composition (real-time PCR) of the faecal gut microbiota with prediction of the ecology of the microbiota at 56 and 84 days (V4 and V5 respectively) from the start of administration; change in stool quality and amount (frequency and consistency); occurrence of neonatal colic, defined according to Rome IV criteria [Castelluzzo]; safety and tolerability of the product; patient prosperity.

### Ethics

This study was conducted in compliance with the ethical principles originated or derived from the Declaration of Helsinki and in accordance with the Good Clinical Practice Guidelines. The investigator submitted all study documents to the relevant Ethics Committee and forwarded to the sponsor a copy of the approval/favourable opinion received from the Ethics Committee (ASL Roma 2: Prot.n°01220672/2018). All parents/guardians of the newborns provided signed informed consent.

### Evaluation of Results

Stool samples were collected within 24 hours prior to each visit.

For each newborn, the number of evacuations was recorded and the colour and consistency of the stool sample was assessed using the Amsterdam Stool Scale (described below).

In addition, physico-chemical analyses of faecal samples, including stercobilinogen, neutral fats, fatty acids, soaps, amides, meat and vegetable fibres, mucose and pH, were carried out on the day of each visit by the Clinical Pathology Laboratory of the Casilino Hospital. In order to ensure optimal preservation conditions, the stool samples were stored in the hospital freezer at −20° C. and sent monthly, with dry ice, by courier charged by the Sponsor, to the Laboratory of Parasitology/Human Microbiome of the Bambino Gesù Paediatric Hospital (San Paolo site) for molecular analysis.

Real-time PCR analyses were carried out to assess the bacterial load of the L. casei DG® strain (according to an already validated and published method; Ferrario) and metagenomic and metabolomic analyses were performed, characterising the gut microbiota before and after administration of the probiotic. In particular, the microbiota was assessed through nucleotide sequence analysis of portions of the gene coding for the bacterial ribosomal subunit 16S rRNA. Metagenomic DNA was extracted from stool samples, quantified and normalised. The V3-V4 hypervariable regions of the bacterial gene coding for 16S rRNA were amplified by PCR and the PCR products were quantified and sequenced using the Illumina MiSeq technique. Bioinformatic sequence analysis was performed for microbial community characterisation, hierarchical clustering, taxonomic analysis and construction of phylogenetic dendrograms with heatmaps.

For metabolomic analysis, metabolites of microbial origin produced as volatile organic compounds (VOCs—chemical categories: short-chain fatty acids, alcohols, ketones, aldehydes, thiols, acids, esters, pyrazine, pyridine, phenols, furans, terpenes, alkanes, alkenes, etc.) were identified and quantified. These compounds were extracted with a GC-MS/SPME (Gas Chromatography coupled to Mass Spectrometry by Solid Phase Micro-Extraction) system. For VOCs extraction, a carboxyl-polydimethylsiloxane (CAR-PDMS) coated fibre (85 μm) was used in the SPME process. Each sample was analysed in triplicate and an average of 100-500 mg was placed in 10 ml glass bottles with the addition of 4-methyl-2-pentanol as an internal standard (IS). Subsequently, stool samples were equilibrated for 10 minutes at 45° C. The fibre was exposed to each sample for 45 minutes before injection into GC-MS (Hewlett Packard 6890 GC) coupled to a 5973C selective mass detector and equipped with a Supelcowax 10 capillary column. Metabolites were detected according to their characteristics. The metabolites were detected according to their retention times (Rt) relative to the pure compound. Chromatograms were integrated and identified by comparison of the fragment patterns with those from the NIST library, followed by manual visual inspection. Quantitative metabolite data were obtained by interpolation of the relative areas with respect to the IS area.

Safety was assessed by monitoring adverse events during the study and measuring the weight, length and head circumference of the neawborns at days 1 (V1), 10 (V2), 28 (V3), 56 (V4) and 84 (V5).

Finally, the assessment of the occurence of colic was carried out according to the ‘Rome IV’ diagnostic criteria (described below) and the stools daily frequency and consistency was monitored by means of a specific diary given to the mother at V1.

### Amsterdam Stool Scale

The scale classifies the amount of stools into four classes: 1: smear, 2: up to 25%, 3:25-50% and 4: >50%.

The stool consistency is classified as: A: watery, B: soft, C: formed and D: hard.

Finally, the stool colour is classified into six classes (I-VI).

### “Rome IV” Diagnostic Criteria for Infant Colic

For clinical purposes, the diagnosis of infant colic must be based on the presence of all the following criteria: The child is less than 5 months old at symptom onset and resolution; recurrent periods of prolonged crying, agitation or irritability that occur without apparent cause and that the parent is unable to prevent or resolve; no evidence of growth deficit, fever or illness. The term ‘agitation’ refers to intermittent vocalisation and is defined as ‘[behaviour] that is not exactly crying, but neither is that of someone who is awake and happy’. Children often alternate between crying and agitation, making it difficult to distinguish the two symptoms.

For research purposes, the diagnosis of infant colic must include the previous criteria, together with both of the following: During a telephone call or one-on-one interview with a researcher or physician, parents report a period of crying or agitation lasting at least 3 hours per day for at least 3 days per week; the duration of crying and agitation, reported as continuous for 24 hours in a selected group of children, confirmed as lasting at least 3 hours, is prospectively quantified through the completion of a daily diary.

### Statistical Analysis

The working hypothesis assumes that L casei DG® (Lactobacillus paracasei CNCMI-1572; LCDG) behaves similarly to L. salivaris which, in an identical study, showed a difference after 28 days [Putignani et al, in submission] of 2.36 Log 10/200 mg stool copies (from 0.04 with SD 0.4 to 2.5 with SD 2.5). In each group (NBW, LBW, VLBW), recognising the intake in the treatment group of 2 billion CFU/day, a sample of 9 patients in each treatment group would give an 80% power to the study, with a two-tailed t-test and 95% significance level in each stratum.

It should be noted that the strain of bacteria L. casei DG® (CNCM I-1572) or L. paracasei DG® (CNCM I-1572) was redeposited on 2 Feb. 2022 as Lacticaseibacillus paracasei DG I-1572 DSM 34154 following the reclassification of the genus Lactobacillus published by Zheng et al. in the scientific journal Int. J. Syst. Evol. Microbiol., 70 (4): 2782-2858, 2020. The two aforementioned designations are interchangeable with each other because they always refer to the same strain of bacteria.

In anticipation of a potential loss at follow-up (10% drop-outs), 20 newborns for each weight stratum were enrolled (of which 10 received active treatment and 10 placebo), for a total of 60 newborns.

Categorical data were represented as counts and percentages, while continuous data were expressed in terms of averages and standard deviations or medians and ranges.

Comparisons of categorical data were performed using the Chi-square test or Fisher's exact test, as appropriate. Continuous data were compared using Student's t-test (for independent or dependent data), Anova's test and Anova for repeated measures, where the appropriate statistical tests determined the normality of the data; in the case of non-normal distribution, non-parametric tests such as Mann Whitney Rank sum test or Wilkoxon Signed Rank test, Kruskal Wallis or Friedman tests were applied. Multivariate regression methods were used if indicated to assess the simultaneous effect of a set of independent variables on a response variable, or to identify potential confounders and/or analyses for temporal data.

The results obtained were considered statistically significant with a P-value <0.05. All analyses were performed with Stata 15 software (StatCorp).

### Results

### Patient Arrangement and Basic Characteristics

Sixty subjects, selected from preterm and term newborns at the Neonatology Unit of the Policlinico Casilino Hospital, were randomised in a 1:1 ratio to the two treatment arms: 30 were treated with LCDG and 30 with placebo′ (FIG. 5).

In the Placebo group, 20 subjects (66.7%) completed the study and 10 (33.3%) did not: 2 (6.7%) subjects withdrew their consent, 1 (3.3%) patient discontinued the study early due to an adverse event (AE), 6 (20.0%) subjects were lost during follow-up (FUP) and 1 (3.3%) subject withdrew due to the need for surgery for hamartoma of the hypopharynx.

In the LCDG group, 26 subjects (86.7%) completed the study and 4 (13.3%) did not; of these, 3 (10.0%) were lost during FUP and 1 (3.3%) died due to ‘disseminated intravascular coagulation’ and ‘septic shock’. With regard to demographic and basic characteristics, the mean age of the mother was 35 years±6 years for the placebo group and 35 years±5 years for the LCDG (treatment) group (P=0.94) and there were no statistically significant differences between the two treatment groups with regard to ethnic group of the mothers, type of breastfeeding, weight and height of the mother, type of diet, smoking status, physical activity, presence of pets and level of education.

With regard to the characteristics of the newborns, they were classified in the study according to their birth weight into 3 groups: normal birth weight (NBW), low birth weight (LBW) and very low birth weight (VLBW). During the study, 12 pairs of twins were born (total of 24 newborns), specifically 14 (46.7%) subjects were included in the LCDG group and 10 (34.5%) in the placebo group (P=0.34). 18 (62.1%) and 17 (56.7%) newborns were female in the placebo and LCDG groups, respectively (P=0.67) and the average birth weight of the newborns was 2050 g±784 g and 2005 g±768 g in the placebo and LCDG groups, respectively (P=0.82). In terms of APGAR score, gestational age and PN/EG ratio (AGA and SGA), there were also no statistically significant differences between the two treatment groups.

L. casei DG (LCDG) was Detected in the Gut from 10 Days after the First Administration and Remained Persistent Until the Last Visit (84 Days).

We analysed 251 stool samples collected from 59 subjects at birth, stratified into VLW (n=20), LW (n=20) and NW (n=19). The LCDG strain was detected in stool samples from 10 days after the first administration (V2) and remained persistent until the last visit (84 days; end-of-study visit), with a slight decrease from the first follow-up time-point (56^{th} study day; 28 days since the last administration of the product) (FIG. 6A). The presence of LCDG was significantly higher in the treatment group than in the control group in samples collected at visits during both the treatment period (at 10 and 28 days) and follow-up (at 56 and 84 days). This evidence was also confirmed by stratifying the samples from each group according to birth weight (NW, LW and VLW). In particular, the comparison between the 3 weight classes (intra-group analysis) in the placebo group showed a statistical difference (p value=0.01) in LCDG concentrations at V2 (10 days after the first administration), whereas in the treatment group, a statistical difference (p value=0.01) in LCDG concentrations between the 3 weight classes occurred at V3 (28 days after the last administration of the product). At the other time points evaluated, no statistically significant difference was observed between the weight classes (FIG. 6B). Furthermore, at day 56, the difference in LCDG concentrations between the treatment and placebo groups was statistically significant for the NW and VLW babies (FIG. 6B).

The Wilcoxon signed-rank test was performed to compare LCDG concentrations at different time-points for each group. In the placebo group, comparisons between V1 and V2 (p-value=0.012), between V1 and V3 (p-value=0.016) and between V1 and V4 (p-value=0.012) were statistically significant. Comparisons between V1 and V2 (p-value=1.5×10−12), V1 and V3 (p-value=5.8×10−9), V1 and V4 (p-value=1.8×10−8), V1 and V5 (p-value=2.0×10−6), V2 and V4 (p-value=1.6×10−3) and between V2 and V5 (p-value=9.7×10−3) were statistically significant for the treatment group (FIG. 7).

Treatment with LCDG was Able to Preserve the Heterogeneity of the Gut Microbiome

To verify whether LCDG treatment was able to preserve the heterogeneity of the gut microbiome, an α- and β-diversity analysis was performed on stool samples, using a metagenomic method targeting 16S-rNA. s

The analysis of the α-diversity of the subjects divided into placebo group and treatment group at each time-point showed that at time-points V2 (10 days) and V3 (28 days), the microbiota of the placebo group appeared to have greater microbial species richness (p≥0.05) than the treatment group. At time-point V4, the α-diversity indexes appear to be equivalent (p≥0.05), whereas at time-point V5, the treatment group has greater richness than the placebo group (p≥0.05) (FIG. 8A). However, there is no statistically significant comparison between the placebo group and the treatment group at the various time-points (p≥0.05).

With regard to the β-diversity analysis, the diversity matrix obtained with the Unweighted UniFrac algorithm, represented as PCoA at the various time-points between the placebo group and the treatment group, showed that there was no statistically significant comparison (p≥0.05) between the two groups at the various time-points. This result was confirmed by PERMANOVA statistical analysis, applied to the distance matrix calculated with the unweighted Unifrac algorithm (FIG. 8B).

Analysis of the distribution of operational taxonomic units (OTUs) at phylum level and the Kruskall Wallis test applied to comparisons at various time-points were performed, which showed no statistically significant differences (p≥0.05) in any of the placebo/treatment comparisons (FIG. 8C).

At the genus level, the only statistically significant comparison between placebo and treatment was observed for time-point V2 (p<0.05); in particular, the Lactobacillus genus was higher in the treatment group (FIG. 8D).

### Administration of LCDG Increases the Concentration of Short-Chain Fatty Acids (SCFAs)

Metabolomic analysis was performed on stool samples (n=85) collected from subjects for whom collection was completed at all follow-up time points (V1 to V5). Stool samples were analysed by gas chromatography combined with mass spectroscopy using a solid-phase microextraction system (GC-MS-SPME).

492 metabolites were identified, quantified and classified into 15 chemical classes: acids, alcohols, aldehydes, alkanes, alkenes, amines, aromatic hydrocarbons, esters, furans, furanones, indoles, ketones, phenols, pyrazines and terpenes. As expected, the metabolic profiles of each sample showed considerable variability between subjects. The raw data matrix was condensed into a matrix of 98 metabolites, mantaining metabolites present in at least 10% of the entire sample set, in order to make the analysis as robust as possible. The entire data analysis was developed on this condensed matrix. A multivariate analysis was conducted using a supervised approach, that is partial least squares discriminant analysis (PLS-DA), for each time point. The greatest clustering of samples according to treatment (placebo/treatment) occurred between time points V2 (10 days) and V4 (56 days), with an increase in SCFA concentration in the treated samples (FIG. 9).

In addition, at each study visit, a physico-chemical evaluation of the stools was performed to assess the presence of neutral fats, acid fats, soaps, starches, meat and vegetable fibres, mucus and pH value. The chi-square test was applied to compare any differences between the two treatment groups and no significant differences (P>0.05) were found for any parameter.

### The Administration of LCDG had an Impact on Both the Ecology and Function of the Microbiota, Modifying the Ecological Niches of Bacteria Residing in the Gut and the Corresponding Functional Profile.

The results obtained with the 3 different approaches, Real-Time PCR, metagenomic and metabolomic, were correlated with each other by means of a Spearman correlation. Time points V2 and V3, which coincide with the administration of the probiotic at 10 and 28 days respectively, were chosen for the correlation analyses, as the metagenomic, metabolomic and Real-Time analyses showed the greatest statistically significant differences between the placebo and treated groups at these time points.

In particular, at time-point V2, Lactobacillus paracasei (CFU/mL) correlated positively (p≤0.05) with the metabolites cyclohexanone, n-decanoic acid and 3,4-dimethyl heptane and with the OTUs Lactobacillus, Granulicatella. Furthermore, several statistically significant positive correlations between OTUs and metabolites can be noted. Those of greatest interest following probiotic intake are SCFAs, including butanoic acid, which shows significant correlations (p≤0.05) with Ruminococcus, Prevotella, Collinsella, Faecalibactarium and Oscillospira. Pentanoic acid shows significant correlations (p≤0.05) with Streptococcus, Granulicatella and Enterococcus, while propionic acid has significant correlations (p≤0.05) with Eggerthella, Bacteroides and Bifidobacterium.

At time-point V3, Lactobacillus paracasei (CFU/mL) was positively correlated (p≤0.05) with 2,3-dimethyl pentanal, 1-hexanol, decanal and dodecanoic acid (FIG. 10A), whereas no significant correlations were found between Lactobacillus paracasei (CFU/mL) and the various OTUs (FIG. 10B). Finally, several statistically significant positive correlations (p≤0.05) between OTUs and metabolites can be noted, in particular, butanoic and propanoic acid show significant correlations (p≤0.05) with Collinsella, while pentanoic acid has a positive correlation with Propionibacterium, Faecalibacterium, Prevotella and Coprococcus (FIG. 10C).

### The Administration of LCDG Did not Reduce the Number of Colics.

According to the ‘Rome IV’ diagnostic criteria, the investigator recorded an episode of colic if the crying was more than 3 hours for at least 3 days a week together with a state of agitation of at least 3 hours a day. During the study, the mothers had to fill out a diary in which they had to keep track of the infant's crying if the crying was longer than 3 hours. At all study visits involved (10, 28, 56 and 84 days after randomisation), no statistically significant differences were found between the two study groups (placebo and Enterolactis Baby). In fact, the number of patients who cried more than 3 hours per day and the average duration of crying in each study group were almost similar at each study visit, with a total number of 8 colics occurring during the study (4 in the placebo group and 4 in the LCDG group).

### Stool Consistency, Amount and Colour

During the clinical trial, stool consistency, amount and colour were assessed using the Amsterdam Stool Scale. The colour and consistency were significantly different between the placebo group and the treatment group at day 10^{th} (V2) and day 56^{th} (V4).

At V2 the p-values were P<0.0001 for the colour of evacuations and P=0.0016 for the consistency of evacuations. Specifically, of the 143 and 221 total evacuations recorded for PTs belonging to the placebo and treatment group, respectively, 37 (25.9%) evacuations for the placebo and 18 (8.1%) evacuations for the treatment group were classified with colour II, and 7 (4.9%) evacuations for the placebo and no evacuations for the treatment group were classified with colour IV. Regarding consistency, there were 80 (55.9%) evacuations for the placebo group and 152 (68.8%) evacuations for the treatment group classified with consistency B, and there were no evacuations for the placebo and 6 (2.7%) and 2 (0.9%) evacuations for the treatment group classified with consistency C/D and D.

At visit 4, 119 evacuations and 183 evacuations occurred for PTs in the placebo and treatment group, respectively, with a statistically significant difference in the colour and consistency between the treatment groups (P=0.007 for the colour of evacuations and P<0.0001 for the consistency of evacuations using the Amsterdam Scale).

With regard to the amount, it was only significant in the samples collected at day 28^{th} (V3) with a total number of 122 and 219 evacuations recorded for the placebo and treatment group respectively.

### Security Assessments

To monitor the growth of the enrolled newborns, weight, length and head circumference parameters were assessed at each study visit. Comparing the treatment and placebo groups, no statistically significant differences were found during the study. Finally, no differences were found in vital signs (with the exception of RR, which was higher in the placebo group than in the treatment group.

Regarding adverse events (AEs), a total of 21 AEs were recorded for the placebo group and 16 AEs for the LCDG group during the study. Of these adverse events, 10 SAEs were recorded for the placebo group and 6 SAEs for the treatment group, and 2 severe AEs for the placebo group and 4 severe AEs for the treatment group.

In the placebo group 10 (47.6%) AE were considered mild, 9 (42.9%) AE of moderate severity and 2 (9.5%) AE of severe severity, whereas in the LCDG group 8 (50.0%) AE were considered mild, 4 (25.0%) AE of moderate severity and 4 (25.0%) AEs of severe severity.

Among the AEs that occurred in the PTs belonging to the placebo group, 3 (14.3%) AE were considered to be unlikely related to the treatment (1 ‘Neonatal respiratory distress syndrome’ and 2 ‘Abdominal pain’) and 1 (4.8%) AE was considered to to be likely related to the treatment (‘Gastroesophageal reflux disease’). In PTs belonging to the LCDG group, 2 (12.5%) AE were considered to be unlikely related to the treatment (‘Disseminated intravascular coagulation’ and ‘Septic shock’, which caused the death of a PT 9 days after visit 2) and 2 (12.5%) AE were considered to be likely related to the treatment (′Necrotising colitis' and ‘Sepsis’).

### Discussion

Certain neonatal clinical trials have studied the action of probiotics in the treatment of intestinal colic, revealing their efficacy in reducing the duration of crying episodes and colic symptoms compared to placebo [Lundelin].

A long-term follow-up study of probiotic treatment during the neonatal period revealed that a lower percentage of subjects in the probiotic-treated group experienced the onset of allergies than those assigned to the placebo group [Denkel].

Furthermore, probiotic administration has been correlated with a lower occurrence of necrotising enterocolitis and a reduced mortality rate in preterm newborns [Lundelin, Strunk, Shane, Costeloe].

According to these findings, probiotics are characterised by an excellent safety profile. Cases of bacteremia caused by bacterial strains contained in probiotics have been recorded, although they are extremely rare and are determined by the presence of severe immunodeficiency [Lundelin, Salminen, Thomas].

However, more evidence is needed on the safety and potential effect of probiotic supplementation in newborns.

Considering the increasing use of lactobacilli in probiotic mixtures, a correct identification and count of LCDG cells in faeces would improve the investigation of the actual stability and persistence of these probiotics in the human gut microbiota [Arioli].

In the present study, for the first time, the safety of LCDG and its ability to positively influence the composition of the microbiota was tested in a special population comprising not only NMW infants but also LW and VLW subjects.

LCDG was detected in the gut from 10 days after the first administration and remained persistent until the last visit (84 days; end of the study), with a slight decrease from the first follow-up point (56th day of the study; 28 days since the last administration of the product). The presence of LCDG was significantly greater in the treatment group than in the control group at V3 (28^{th} days; last day of treatment) for NW and VLW babies.

Analysing the probiotic strains in stool samples, although considering the alpha and beta diversity analysis the change in the entire microbiota profile would appear to be time-dependent and unaffected by treatment (demonstrating that treatment is able to preserve the heterogeneity of the gut microbiome), at the genus level, according to the primary analysis, administration of the probiotic causes a statistically significant increase in the genus Lactobacillus spp 10 days after the start of administration. This increase is also detected at later times, but becomes non-statistically significant.

With regard to metabolomic analysis, as expected, the metabolic profiles of each sample showed considerable variability between subjects. PLS-DA analysis identified butyric acid and propanoic acid (SCFA) metabolites as the main contributors to clustering between the placebo group and the treated group, with an increase in their concentration in the samples collected from the treatment group.

All these results highlighted and confirmed the ability of LCDG to survive the gastrointestinal passage while preserving the heterogeneity of the gut microbiota. These results are consistent with the conclusions of studies conducted in healthy paediatric (3-12 years) [Radicioni] or adult populations [Drago].

Furthermore, the results obtained with the 3 different approaches (real-time PCR, metagenomics and metabolomics) were correlated with each other by means of a Spearman correlation.

Integration of the data obtained from these approaches shows that the administration of the probiotic LCDG has an impact on both the ecology and function of the microbiota, modifying the ecological niches of bacteria residing in the gut and the corresponding functional profile. In particular, some SCFAs, such as butanoic and propionic acid, showed a positive correlation with certain beneficial bacteria, such as Faecalibactarium, Oscillospira, Eggerthella, Bacteroides and Bifidobacterium. These molecules showed the ability to influence nutrient adsorption [Heimann]. In addition, a positive correlation (p≤0.05) between Lactobacillus paracasei (CFU/mL) and cyclohexanone, n-decanoic acid and 3,4-dimethyl heptane was found at test day 28.

Stool consistency, amount and colour were also recorded during the clinical trial. Amount was only significant in samples collected at day 28^{th}, whereas colour and consistency were significantly different at day 10^{th} and day 56^{th} (4 weeks after end of administration).

On the other hand, no difference was observed in the occurence of colic according to ‘Rome IV’ diagnostic criteria, but these results should perhaps be analysed considering that both treatment and total study duration may be insufficient to see an improvement in this parameter. For example, Partty et al [Partty], prolonging probiotic supplementation for up to 2 months (twice the duration of this study), with a follow-up period of up to 1 year, demonstrated that it can alleviate symptoms associated with crying and agitation in preterm newborns.

Regarding safety, as mentioned above, no differences in vital signs were found (except for the RR, which was higher in the placebo group than in the treatment group).

During the study, weight, length and head circumference parameters were also assessed at each study visit to monitor the growth of the enrolled newborns. Comparing the treatment and placebo groups, no statistically significant differences were found during the study. Although a previous article has shown that a commercially prepared symbiotic solution containing a combination of probiotics and prebiotics is able to improve weight and head circumference in preterm newborns [Guney], other studies have shown data consistent with our results (i.e. no difference between the treatment and placebo groups) [Vlieger], particularly for the weight parameter [Indrio, Underwood]. Weight gain in newborns is influenced by many co-existing pathologies and the availability of total parental supplementation and/or breast milk fortification [Moni], which may have influenced the weight trend in both groups in that study.

Recently, Matin et al [Matin] published the results of a study involving the oral administration of probiotics (1.5×10⁹ CFU/g Lacticaseibacillus paracasei subsp. paracasei) to breastfeeding mothers or their very low birth weight (VLBW) newborns to test changes in total serum bilirubin (TSB) level and weight gain of the newborns. The administration of selected probiotics reduced the newborns TSB level, but had no significant effect on weight gain after the first week of intervention.

Furthermore, based on the safety and tolerability profile analysed, as well as the assessment of severe AEs recorded in both study groups, the test product showed to be safe and well-tolerated as placebo in this study.

### Limits and Future Plans

Overall, the results of this study confirmed the product's high safety profile even for a vulnerable population such as NBW, LBW and VLBW newborns. Although LDCG confirmed the ability to survive in the gastrointestinal tract influencing the gut microbiota and SCFAs composition, considering the key role of the gut microbiota in nutrient absorption and, consequently, nweborn growth, it will be useful in the future to analyse the long-term effect of the product by considering a follow-up of at least one year.

Another interesting aspect that has not been fully emphasised in this clinical trial is the potential impact of the product on the microbiome and serum metabolome under different nutritional conditions. Indeed, nutrition in early childhood determines the development of the gut microbiota. Unlike breastfeeding, artificial milk feeding has been shown to impact the gut microbiota and serum metabolome towards a more unfavourable state. Recently, another Lactobacillus paracasei strain, while not canceling the impact of artificial milk feeding, has been shown to have an impact on the microbiome and serum metabolome that may mitigate some unfavourable metabolic impacts of artificial milk feeding [Lee].

### FIGURES

FIG. 5. Flow chart of the study design. 60 participants were enrolled for the clinical trial and randomised into one of the administration groups in a 1:1 ratio. 20 and 26 completed the study in the placebo and treatment group, respectively. 20 and 26 subjects completed the study in the placebo and treatment group, respectively.

FIG. 6. L. casei DG (LCDG) was detected in the gut from 10 days after the first administration and remained persistent until the last visit (84 days).

A) Box plots show LCDG concentrations for each follow-up time-point, differentiated between placebo and treatment; B) Data were stratified according to subjects' birth weight: VLW (very low weight), LW (low weight), NW (normal weight). Median, twenty-fifth percentile, seventy-fifth percentile, minimum and maximum values of L. casei DG concentration (CFU/mL) were shown for each group. The upper bars represent statistically significant comparisons (p-value<0.05).

FIG. 8. LCDG treatment was able to preserve the heterogeneity of the gut microbiome. (A) α diversity analysis between the placebo group and the treatment group at days 10, 28, 56 and 84 (V2, V3, V4 and V5 respectively). B) β-diversity analysis. The diversity matrix was obtained using the Unweighted UniFrac algorithm, represented as PCoA at the various time points between the placebo group and the treatment group. C) Histograms of OTUs distribution at phylum level and Kruskal Wallis test applied to comparisons at various time-points. D) Histograms showing the relative abundance of Lactobacillus spp. at all time-points.

FIG. 9. The administration of LCDG increased the concentration of short-chain fatty acids (SCFAs). Metabolite analysis is shown as Partial Least Squares Discriminant Analysis (PLS-DA; left: score graph; right: VIP scores) comparing placebo with treatment at each time point: A) V1; B) V2; C) V3; D) V4; E) V5.

FIG. 10. The administration of LCDG had an impact on both the ecology and function of the microbiota, modifying the ecological niches of bacteria residing in the gut and the corresponding functional profile. Correlation analysis between Lactobacillus paracasei, metabolites and OTUs at 28 days (V3; end of treatment). A) heatmap of correlation between metabolites and Lactobacillus paracasei (CFU/mL); B) heatmap of correlation between OTUs and L. paracasei (CFU/mL); C) heatmap of correlation between OTUs and metabolites.

1. A method of treating and/or preventing gastro-intestinal disorders of an inflammatory and/or functional nature in a subject in need thereof with a strain of bacteria said disorders being selected from the group consisting of: neonatal colic, intestinal colic, chronic enterocolitis or chronic enterocolitis in preterm newborns, necrotizing enterocolitis, sepsis, irritable bowel syndrome (IBS), IBS with prevalent constipation or constipated bowel, IBS with prevalent diarrhoea or diarrhoeic bowel, IBS with alternating bowel, unclassified IBS, chronic inflammatory bowel disease (IBD), Crohn's disease, ulcerative rectocolitis, asthma, obesity, type 1 diabetes, atopic dermatitis, multiple sclerosis, cancer, autism, allergies, immune-mediated, autoimmune disorders, selected from coeliac disease, Graves' disease, rheumatoid arthritis, Hashimoto's thyroiditis, diabetes mellitus type 1, systemic lupus erythematosus (lupus), vasculitis, Addison's disease, polymyositis, Sjögren's syndrome, progressive systemic sclerosis, glomerulonephritis (inflammation of the kidneys), infertility and support of body weight gain in said subject;
wherein said strain of bacteria belongs to the species Lactobacillus paracasei and is selected from the group consisting of: Lactobacillus paracasei DG® CNCM I-1572, Lactobacillus paracasei LPC-S01 DSM 26760, and a mixture thereof; and
wherein said subject is a newborn subject in the first four weeks of life, said method comprising
administering to said subject a therapeutically effective amount of said strain of bacteria.

2. The method according to claim 1, wherein said subject is aged from 1 month to less than or equal to 12 months.

3. The method according to claim 1, wherein said strain is a viable strain of bacteria or a derivative of said strain of bacteria, the derivative of said strain bacteria being selected from the group consisting of: tyndallized bacterial strain, sonicated bacterial strain, bacterial strain inactivated by radiation lysed bacterial strain, bacterial homogenate and an extract or wall fraction of the bacterial strain.

4. The method according to claim 1, wherein said disorders are selected from the group consisting of: neonatal colic, intestinal colic, chronic enterocolitis or chronic enterocolitis in preterm newborns, necrotizing enterocolitis, sepsis, irritable bowel syndrome (IBS), IBS with prevalent constipation or constipated bowel, IBS with prevalent diarrhoea or diarrhoeic bowel, IBS with alternating bowel, unclassified IBS, chronic inflammatory bowel disease (IBD), Crohn's disease and ulcerative rectocolitis.

5. The method according to claim 1, wherein said mixture consists of a strain of bacteria belonging to the species Lactobacillus paracasei which is selected from the group consisting of: Lactobacillus paracasei DG® CNCM I-1572, Lactobacillus paracasei LPC-S01 DSM 26760, and a mixture thereof; and at least one food grade or pharmaceutical grade additive and/or excipient.

6. The method according to claim 5, wherein said mixture consists of: a strain of bacteria belonging to the species Lactobacillus paracasei selected from the group consisting of: Lactobacillus paracasei DG® CNCM I-1572, Lactobacillus paracasei LPC-S01 DSM 26760, and a mixture thereof; and
at least one further strain of bacteria selected from the group consisting of:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234,
Bifidobacterium bifidum BbfIBS01 DSM 32708; and
a mixture thereof.

7. The method according to claim 5, wherein said mixture further comprises at least one further active component selected from the group consisting of:
at least one vitamin from the group A, B, C, D, E and/or K;
one or more antioxidant substances, selected from the group consisting of: glutathione, polyphenols such as resveratrol and trans-resveratrol, coenzyme Q10, astaxanthin and lycopene;
one or more plant substances, selected from the group consisting of: botanicals, or extracts thereof, with an intestinal relaxing action, selected from valerian, passion flower, lemon balm, hawthorn and camomile;
minerals or salts thereof selected from the group consisting of zinc, selenium, magnesium and potassium;
one or more monounsaturated fatty acids, selected from the group consisting of omega 9, and/or polyunsaturated fatty acids, omega 3 and omega 6;
one or more immunostimulant substances, anti-diarrhoeal substances, and/or nutrients;
at least one prebiotic, selected from group (II) consisting of: inulin, fructo-oligosaccharide (FOS), galacto-oligosaccharide (GOS), xylitol-oligosaccharide (XOS), guar gum, lactoferrin, and a mixture thereof;
and a mixture thereof.

8. The method according to claim 3, wherein said bacterial strain is inactivated by gamma radiation.

9. The method according to claim 3, wherein said bacterial strain is a tyndallized bacterial strain

10. The method according to claim 7, wherein
said at least one vitamin is a group B vitamin and/or vitamin D; and
said at least one prebiotic is inulin.

11. The method according to claim 1, wherein said disorders are selected from the group consisting of: neonatal colic, intestinal colic, chronic enterocolitis or chronic enterocolitis in preterm newborns and necrotizing enterocolitis.

12. The method according to claim 5, wherein said disorders are selected from the group consisting of: neonatal colic, intestinal colic, chronic enterocolitis or chronic enterocolitis in preterm newborns and necrotizing enterocolitis.
